(19) United States
(12) Patent Application Publication
(10) Pub. No.: US 2024/0100132 A1
(43) Pub. Date: Mar. 28, 2024

(54) MODULATION OF AAV-BASED GENE EXPRESSION

(71) Applicant: Prevail Therapeutics, Inc., New York, NY (US)

(72) Inventors: Benjamin Shykind, New York, NY (US); Asa Abeliovich, New York, NY (US); E. David Litwack, New York, NY (US); Kumar Anindya Sen, Revere, MA (US)

(73) Assignee: Prevail Therapeutics, Inc., New York, NY (US)

(21) Appl. No.: 18/273,845

(22) PCT Filed: Jan. 24, 2022
(86) PCT No.: PCT/US2022/013476

§ 371 Date: Jul. 24, 2023

Related U.S. Application Data
(60) Provisional application No. 63/141,110, filed on Jan. 25, 2021.

(51) Int. Cl. A61K 38/47; A61K 31/712; A61K 48/00; A61P 1/16; C12N 15/113; C12N 15/86

(52) U.S. Cl. CPC A61K 38/47; A61K 31/712; A61K 48/0058; A61P 1/16; C12N 15/113; C12N 15/86; C12Y 302/01021; C12N 2310/11; C12N 2310/315; C12N 2310/321; C12N 2310/3231; C12N 2310/341; C12N 2750/14143

(57) ABSTRACT

Aspects of the disclosure relate to compositions and methods for positively or negatively regulating the expression of a gene therapeutic (e.g., a therapeutic protein expressed from an AAV vector). The disclosure is based, in part, on certain nucleic acids, for example antisense oligonucleotides (ASOs), configured to bind specific regions of an expression cassette (or an mRNA transcribed from such an expression cassette).

### RELATED APPLICATIONS

This Application is a national stage filing under 35 U.S.C. § 371 of international PCT application PCT/US2022/013476, filed Jan. 24, 2022, which claims priority under 35 U.S.C. § 119(e) to U.S. provisional patent application, U.S. Ser. No. 63/141,110, filed Jan. 25, 2021, the entire contents of each of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 17, 2023, is named P109470014US03-SEQ-KZM and is 29,845 bytes in size.

### BACKGROUND

The ability to regulate the expression of a therapeutic transgene introduced into a patient via AAV transduction would transform the field of gene therapy. The biology underpinning the current viral approach to gene therapy ensures a long-term expression of the therapeutic transgene. This continuous expression of the therapeutic gene is appropriate for many indications, especially those resulting from a loss of function gene mutation in the genome of the patient. However, several scenarios exist where it would be highly desirable from a therapeutic dosing perspective, or from a safety perspective, to have the ability to turn therapeutic gene expression up or down, on or off; this invention describes multiple methods by which to achieve such control of gene expression through the use of specific ASOs.

### SUMMARY

Aspects of the disclosure relate to compositions and methods for positively or negatively regulating the expression of a gene therapeutic (e.g., a therapeutic protein expressed from an AAV vector), through the use of certain nucleic acids, for example antisense oligonucleotides (ASOs). In some embodiments, methods described by the disclosure allow for modulation of gene expression from expression cassettes comprised of generic, widely-used, cis-acting DNA or RNA regulatory elements, or from expression cassettes bearing cis-acting DNA or RNA elements, through the interaction of the nucleic acids (e.g., ASOs) with elements in the expression cassette or a mRNA transcribed from such an expression cassette.

Accordingly, in some aspects, the disclosure provides a method for modulating expression of a transgene in a cell, the method comprising contacting a cell containing an rAAV vector comprising a transgene flanked by AAV inverted terminal repeats (ITRs) with one or more antisense oligonucleotides (ASOs) that specifically bind to at least one of the AAV ITRs, wherein binding of the one or more ASOs to the AAV ITR results in altered expression of the transgene relative to a cell that does not contain the one or more ASOs.

In some embodiments, each of the one or more ASOs ranges from about 10 nucleotides to about 30 nucleotides in length. In some embodiments, each of the ASOs comprises one or more chemical modification. In some embodiments, the one or more chemical modifications are selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs).

In some embodiments, an AAV ITR is an AAV2 ITR. In some embodiments, an AAV2 ITR comprises a nucleic acid sequence that is at least 90% identical to the nucleic acid sequence set forth in SEQ ID NO: 1. In some embodiments, an AAV2 ITR consists of the nucleic acid sequence set forth in SEQ ID NO: 1 or the complement thereof. In some embodiments, an ASO binds to at least three contiguous nucleotides of an AAV ITR.

In some embodiments, at least one ASO comprises a nucleic acid sequence that is at least 90% identical to the sequence set forth in any one of SEQ ID NOs: 2-8, or a complement thereof. In some embodiments, at least one ASO comprises a nucleic acid sequence that is at least 90% identical to the sequence set forth in any one of SEQ ID NOs: 2-8, or a complement thereof, and each of the at least one ASO comprises one or more chemical modification selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs).

In some embodiments, altered expression is increased expression of the transgene (e.g., increased expression relative to a cell that does not comprise the one or more ASOs). In some embodiments, altered expression is decreased expression of the transgene (e.g., decreased expression relative to a cell that does not comprise the one or more ASOs).

In some embodiments, a cell is a mammalian cell. In some embodiments, a mammalian cell is a human cell. In some embodiments, a cell is in a subject.

In some embodiments, a transgene is a therapeutic protein. In some embodiments, a therapeutic protein is β-glucocerebrosidase (GBA). In some embodiments, GBA is encoded by a codon-optimized nucleic acid sequence. In some embodiments, a transgene encoding GBA comprises the nucleic acid sequence set forth in SEQ ID NO: 40 or the complement thereof. In some embodiments, an rAAV vector comprises the nucleic acid sequences set forth in SEQ ID NOs: 1, 9, 25, 40, 51, and 80.

In some aspects, the disclosure provides a method for modulating expression of a transgene in a cell, the method comprising contacting a cell containing an rAAV vector comprising a transgene with one or more antisense oligonucleotides (ASOs) that specifically bind to a transcriptional control region sequence of the transgene, wherein binding of the one or more ASOs to the transcriptional control region sequence results in altered expression of the transgene relative to a cell that does not contain the one or more ASOs.

In some embodiments, each of the one or more ASOs ranges from about 10 nucleotides to about 30 nucleotides in length. In some embodiments, each of the ASOs comprises one or more chemical modification. In some embodiments, all the nucleobases and/or the entire backbone of the ASO are modified. In some embodiments, the one or more chemical modifications are selected from a nucleobase modification or a backbone modification. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs).

In some embodiments, a transcriptional control region sequence comprises an enhancer sequence and/or a promoter sequence. In some embodiments, an enhancer sequence is a cytomegalovirus (CMV) enhancer sequence and/or a promoter sequence is a chicken beta-actin (CBA) promoter sequence. In some embodiments, a CMV enhancer sequence comprises a nucleic acid sequence that is at least 90% identical to the nucleic acid sequence set forth in SEQ ID NO: 9 or the complement thereof. In some embodiments, a chicken beta-actin (CBA) promoter sequence comprises a nucleic acid sequence that is at least 90% identical to the nucleic acid sequence set forth in SEQ ID NO: 25 or the complement thereof.

In some embodiments, an ASO binds to at least three contiguous nucleotides of a transcriptional control region sequence.

In some embodiments, at least one ASO comprises a nucleic acid sequence that is at least 90% identical to the sequence set forth in any one of SEQ ID NOs: 10-24 and 26-39, or a complement thereof. In some embodiments, at least one ASO comprises a nucleic acid sequence that is at least 90% identical to the sequence set forth in any one of SEQ ID NOs: 10-24 and 26-39, or a complement thereof, and each of the at least one ASO comprises one or more chemical modification selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs).

In some embodiments, altered expression is increased expression of the transgene (e.g., increased expression relative to a cell that does not comprise the one or more ASOs). In some embodiments, altered expression is decreased expression of the transgene (e.g., decreased expression relative to a cell that does not comprise the one or more ASOs).

In some embodiments, a cell is a mammalian cell. In some embodiments, a mammalian cell is a human cell. In some embodiments, a cell is in a subject.

In some embodiments, a transgene is a therapeutic protein. In some embodiments, a therapeutic protein is β-glucocerebrosidase (GBA). In some embodiments, GBA is encoded by a codon-optimized nucleic acid sequence. In some embodiments, a transgene encoding GBA comprises the nucleic acid sequence set forth in SEQ ID NO: 40 or the complement thereof. In some embodiments, an rAAV vector comprises the nucleic acid sequences set forth in SEQ ID NOs: 1, 9, 25, 40, 51, and 80.

In some aspects, the disclosure provides a method for modulating expression of a transgene in a cell, the method comprising contacting a cell containing an rAAV vector comprising a transgene with one or more antisense oligonucleotides (ASOs) that specifically bind to a protein coding region of an mRNA transcribed from the transgene, wherein binding of the one or more ASOs to the protein coding region results in altered expression of the transgene relative to a cell that does not contain the one or more ASOs.

In some embodiments, each of the one or more ASOs ranges from about 10 nucleotides to about 30 nucleotides in length. In some embodiments, each of the ASOs comprises one or more chemical modification. In some embodiments, the one or more chemical modifications are selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs). In some embodiments, an ASO comprises a gapmer structure. In some embodiments, an ASO binds to at least three contiguous nucleotides of the protein coding region.

In some embodiments, at least one ASO comprises a nucleic acid sequence that is at least 90% identical to the sequence set forth in any one of SEQ ID NOs: 41-50, any one of SEQ ID NOs: 91-95, or any one of SEQ ID NOs: 106-110, or a complement thereof.

In some embodiments, a protein coding region encodes a β-glucocerebrosidase (GBA) protein. In some embodiments, a protein coding region comprises a nucleic acid sequence that is at least 90% identical to the nucleic acid sequence set forth in SEQ ID NO: 40 or the complement thereof.

In some embodiments, altered expression is increased expression of the transgene (e.g., increased expression relative to a cell that does not comprise the one or more ASOs). In some embodiments, altered expression is decreased expression of the transgene (e.g., decreased expression relative to a cell that does not comprise the one or more ASOs). In some embodiments, decreased expression of a transgene results from RNaseH-mediated degradation of mRNA transcripts bound by the one or more ASOs.

In some embodiments, a cell is a mammalian cell. In some embodiments, a mammalian cell is a human cell. In some embodiments, a cell is in a subject.

In some embodiments, an rAAV vector comprises the nucleic acid sequences set forth in SEQ ID NOs: 1, 9, 25, 40, 51, and 80.

In some aspects, the disclosure provides a method for modulating expression of a transgene in a cell, the method comprising contacting a cell containing an rAAV vector comprising a transgene with one or more antisense oligonucleotides (ASOs) that specifically bind to a woodchuck post-translational regulatory element (WPRE) of an mRNA transcribed from the transgene, wherein binding of the one or more ASOs to the WPRE results in altered expression of the transgene relative to a cell that does not contain the one or more ASOs.

In some embodiments, each of the one or more ASOs ranges from about 10 nucleotides to about 30 nucleotides in length. In some embodiments, each of the ASOs comprises one or more chemical modification. In some embodiments, the one or more chemical modifications are selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs).

In some embodiments, a WPRE comprises a nucleic acid sequence that is at least 90% identical to the nucleic acid sequence set forth in SEQ ID NO: 51 or the complement thereof. In some embodiments, an ASO binds to at least three contiguous nucleotides of the WPRE sequence.

In some embodiments, at least one ASO comprises a nucleic acid sequence that is at least 90% identical to the sequence set forth in any one of SEQ ID NOs: 52-79, any one of SEQ ID NOs: 96-100, or any ne of SEQ ID NOs: 111-115, or a complement thereof.

In some embodiments, altered expression is increased expression of the transgene (e.g., increased expression relative to a cell that does not comprise the one or more ASOs). In some embodiments, altered expression is decreased expression of the transgene (e.g., decreased expression relative to a cell that does not comprise the one or more ASOs).

In some embodiments, a cell is a mammalian cell. In some embodiments, a mammalian cell is a human cell. In some embodiments, a cell is in a subject.

In some embodiments, a transgene is a therapeutic protein. In some embodiments, a therapeutic protein is β-glucocerebrosidase (GBA). In some embodiments, GBA is encoded by a codon-optimized nucleic acid sequence. In some embodiments, a transgene encoding GBA comprises the nucleic acid sequence set forth in SEQ ID NO: 40 or the complement thereof. In some embodiments, an rAAV vector comprises the nucleic acid sequences set forth in SEQ ID NOs: 1, 9, 25, 40, 51, and 80.

In some aspects, the disclosure provides a method for modulating expression of a transgene in a cell, the method comprising contacting a cell containing an rAAV vector comprising a transgene with one or more antisense oligonucleotides (ASOs) that specifically bind to a polyadenylation element of an mRNA transcribed from the transgene, wherein binding of the one or more ASOs to the polyadenylation element results in altered expression of the transgene relative to a cell that does not contain the one or more ASOs.

In some embodiments, each of the one or more ASOs ranges from about 10 nucleotides to about 30 nucleotides in length. In some embodiments, each of the ASOs comprises one or more chemical modification. In some embodiments, the one or more chemical modifications are selected from a nucleobase modification or a backbone modification. In some embodiments, all the nucleobases and/or the entire backbone of the ASO are modified. In some embodiments, a nucleobase modification comprises a 2′-O-methyl (2′OMe) modification. In some embodiments, a backbone modification comprises a phosphorothioate linkage. In some embodiments, an ASO comprises one or more locked nucleic acids (LNAs). In some embodiments, an ASO comprises a gapmer structure. In some embodiments, an ASO binds to at least three contiguous nucleotides of a polyadenylation element.

In some embodiments, a polyadenylation element comprises the nucleic acid sequence set forth in SEQ ID NO: 80 or the complement thereof. In some embodiments, at least one ASO comprises a nucleic acid sequence that is at least 90% identical to the sequence set forth in any one of SEQ ID NOs: 81-90, any one of SEQ ID NOs: 101-104, or any one of SEQ ID NOs: 116-120, or a complement thereof.

In some embodiments, altered expression is increased expression of the transgene (e.g., increased expression relative to a cell that does not comprise the one or more ASOs). In some embodiments, altered expression is decreased expression of the transgene (e.g., decreased expression relative to a cell that does not comprise the one or more ASOs). In some embodiments, decreased expression of a transgene results from RNaseH-mediated degradation of mRNA transcripts bound by the one or more ASOs.

In some embodiments, the expression of the transgene is altered (i.e. increased or decreased), irrespective of the nature of the expressed transgene. In some embodiments, the one or more ASOs are delivered to the cell at the same time of transgene transfection. In some embodiments, the one or more ASOs are delivered to the cell several hours, for example 3 hours, after the cell is transfected with the plasmid comprising the rAAV vector encoding the transgene. In some embodiments, the one or more ASOs are delivered to the cell several weeks after the cell is transfected with the plasmid comprising the rAAV vector encoding the transgene.

In some embodiments, a cell is a mammalian cell. In some embodiments, a mammalian cell is a human cell. In some embodiments, a cell is in a subject.

In some embodiments, a transgene is a therapeutic protein. In some embodiments, a therapeutic protein is β-glucocerebrosidase (GBA). In some embodiments, GBA is encoded by a codon-optimized nucleic acid sequence. In some embodiments, a transgene encoding GBA comprises the nucleic acid sequence set forth in SEQ ID NO: 40 or the complement thereof. In some embodiments, an rAAV vector comprises the nucleic acid sequences set forth in SEQ ID NOs: 1, 9, 25, 40, 51, and 80.

In some aspects, the disclosure provides an isolated nucleic acid comprising the sequence set forth in any one of SEQ ID NOs: 2-8, 10-24, 26-39, 41-50, 52-79, 81-120, or a complement thereof. In some embodiments, an isolated nucleic acid comprises one or more chemical modifications. In some embodiments, the one or more chemical modifications comprises a 2′-O-methyl (2′OMe) modification, a phosphorothioate linkage, a locked nucleic acid (LNA), or any combination of the foregoing. In some embodiments, the isolated nucleic acid is an antisense oligonucleotide (ASO). In some embodiments, all the nucleobases and/or the entire backbone of the ASO are modified. In some embodiments, the isolated nucleic acid has a gapmer structure.

### BRIEF DESCRIPTION OF DRAWINGS

FIG. 1 shows the effects of ASOs directed against GBA on GBA expression in HEK293T cells transfected with plasmid encoding GBA and with the indicated ASO. Cells were harvested after 72 hours and GBA expression was quantified using qRT-PCR.

FIG. 2 shows the effects of ASOs directed against WPRE on GBA expression in HEK293T cells transfected with plasmid encoding GBA and with the indicated ASO. Cells were harvested after 72 hours and GBA expression was quantified using qRT-PCR.

FIG. 3 shows the effects of ASOs directed against BGH PolyA on GBA expression in HEK293T cells transfected with plasmid encoding GBA and with the indicated ASO. Cells were harvested after 72 hours and GBA expression was quantified using qRT-PCR.

FIG. 4 shows the effects of ASO PolyA ASO-2 on Trem2 expression in HEK293T cells transfected with plasmid encoding Trem2 and with the indicated ASO. Cells were harvested after 72 hours and GBA expression was quantified using qRT-PCR.

FIG. 5 shows the effects of ASOs in sequential transfection on GBA expression. HEK293T cells were transfected with a plasmid encoding GBA expression. After 3 hours, plasmid transfection mixture was removed and cells were transfected with indicated ASO. Cells were harvested after 72 hours and GBA expression was quantified using qRT-PCR.

FIG. 6 shows ASO target sequences for the PR001 construct.

FIG. 7 shows GBA mRNA level in liver of mice received AAV-GBA infusion followed by GBA ASO 1, WPRE ASO 2, or PolyA ASO2 treatment.

### DETAILED DESCRIPTION

In some aspects, the disclosure relates to compositions and methods for modulating (e.g., positively or negatively regulating) the expression of a gene therapeutic (e.g., a therapeutic protein expressed from an AAV vector), through the use of certain nucleic acids, for example antisense oligonucleotides (ASOs). In some embodiments, nucleic acids described herein (e.g., ASOs) modulate gene expression from expression cassettes comprised of generic, widely-used, cis-acting DNA or RNA regulatory elements, from expression cassettes bearing cis-acting DNA or RNA elements, or mRNAs transcribed from such expression cassettes.

### Isolated Nucleic Acids

An isolated nucleic acid may be DNA or RNA. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The disclosure relates, in part, to isolated nucleic acids (e.g., artificial or synthetic isolated nucleic acids) comprising a region of complementarity with a target nucleotide sequence, such as a sequence from an rAAV vector, expression construct, or an mRNA transcribed from an expression construct. In some embodiments, an isolated nucleic acid is an antisense oligonucleotide (ASO).

### Antisense Oligonucleotides

As used herein, the term, “antisense nucleic acid” or “ASO” refers to a nucleic acid that has sequence complementarity to a target sequence and is specifically hybridizable, e.g., under stringent conditions, with a nucleic acid having the target sequence. An antisense nucleic acid is specifically hybridizable when binding of the antisense nucleic acid to the target nucleic acid is sufficient to produce complementary based pairing between the antisense nucleic acid and the target nucleic acid, and there is a sufficient degree of complementarity to avoid non-specific binding of the antisense nucleic acid to non-target nucleic acid under conditions in which specific binding is desired, e.g., under physiological conditions in the case of in vivo assays or therapeutic treatment, and in the case of in vitro assays, under conditions in which the assays are performed. An ASO may comprise one or more DNA nucleobases, one or more RNA nucleobases, or a combination of DNA and RNA nucleobases.

Complementary refers to the capacity for precise pairing between two nucleotides. For example, if a nucleotide at a certain position of an antisense nucleic acid is capable of hydrogen bonding with a nucleotide at the corresponding position of a target nucleic acid (e.g., target nucleic acid sequence), then the antisense nucleic acid and target nucleic acid are considered to be complementary to each other at that position. The antisense nucleic acid and target nucleic acid are complementary to each other when a sufficient number of corresponding positions in each molecule are occupied by nucleotides that can hydrogen bond with each other through their bases. Thus, “complementary” is a term that is used to indicate a sufficient degree of complementarity or precise pairing such that stable and specific binding occurs between the antisense nucleic acid and target nucleic acid. However, it should be appreciated that 100% complementarity is not required. For example, in some embodiments, an antisense nucleic acid (e.g., an oligonucleotide, such as an ASO) may be at least 80% complementary to (e.g., at least 85%, 90%, 91%, 92%, 93%, 940%, 95%, 96%, 97%, 98%, 99% or 100% complementary to) the consecutive nucleotides of a target nucleic acid sequence.

In some embodiments, an antisense nucleic acid (ASO) is used that has a region of complementarity that is perfectly complementary (e.g., 100% complementary) to a portion of a target nucleic acid (e.g., a target sequence of an rAAV vector, a target sequence of an expression construct, an mRNA sequence transcribed from an expression construct, etc.). In some embodiments, an antisense nucleic acid oligonucleotide comprises a region of complementarity that is complementary with the sequence as set forth in any one of SEQ ID NO: 1, 9, 25, 40, 51, and 80. The region of complementarity of the antisense nucleic acid may be complementary with at least 3, at least 4, at least 5, at least 6, e.g., at least 7, at least 8, at least 9, at least 10, at least 15, at least 16, at least 17, at least 18, at least 19, at least 20, at least 21, at least 22, at least 23, at least 24, at least 25, or more consecutive nucleotides of a target nucleic acid sequence. In addition, to minimize the likelihood of off-target effects, an ASO may be designed to ensure that it does not have a sequence (e.g., of 5 or more consecutive nucleotides) that is complementary with an off-target nucleic acid.

However, it should be appreciated that in some embodiments, an antisense nucleic acid may be used that has less than 100% sequence complementarity with a target nucleic acid. Thus, it is understood in the art that a complementary nucleotide sequence need not be 100% complementary to that of its target to be specifically hybridizable. For example, in some embodiments, an isolated nucleic acid comprises at least 1, 2, 3, 4, 5, 6, 7, 8, 9, or 10 mismatches with its target sequence. In some embodiments, a complementary nucleic acid sequence for purposes of the present disclosure is specifically hybridizable when binding of the sequence to the target nucleic acid produces the desired alterations in gene expression (e.g., increased expression or translation of a gene product or decreased expression or translation of a gene product) to occur and there is a sufficient degree of complementarity to avoid non-specific binding to non-target nucleic acids under conditions in which avoidance of non-specific binding is desired, e.g., under physiological conditions in the case of in vivo assays or therapeutic treatment, and in the case of in vitro assays, under conditions in which the assays are performed under suitable conditions of stringency.

Sequence identity, including determination of sequence complementarity for nucleic acid sequences, may be determined by sequence comparison and alignment algorithms known in the art. To determine the percent identity of two nucleic acid sequences, the sequences are aligned for optimal comparison purposes (e.g., gaps can be introduced in the first sequence or second sequence for optimal alignment). The nucleotides at corresponding nucleotide positions are then compared. When a position in the first sequence is occupied by the same residue as the corresponding position in the second sequence, then the molecules are identical at that position. In some embodiments, the percent identity between the two sequences is a function of the number of identical positions shared by the sequences (e.g., % homology=# of identical positions/total # of positions×100), optionally penalizing the score for the number of gaps introduced and/or length of gaps introduced.

In some embodiments, oligonucleotides of the disclosure (e.g., ASOs) have a length in a range of 5 to 40 nucleotides, 5 to 30 nucleotides, 10 to 30 nucleotides, 10 to 25 nucleotides, or to 25 nucleotides. In some embodiments of the disclosure, oligonucleotides have a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more. In some embodiments, the oligonucleotide comprises a region of complementarity that is complementary with a region within 5, 10, 15, 25, or more nucleotides of an rAAV vector sequence, expression cassette sequence, or sequence of an mRNA transcribed from an expression cassette or rAAV vector described herein.

In some embodiments, antisense nucleic acids (e.g., oligonucleotides) are provided in a homogeneous preparation, e.g., in which at least 85%, at least 90%, at least 95%, or at least 99% of the oligonucleotides are identical. In some embodiments, a composition described by the disclosure is heterogeneous with respect to ASOs (e.g., a composition may comprise 2, 3, 4, 5, 6, 7, or more different sequences of ASOs).

Antisense nucleic acids of the disclosure may be modified to achieve one or more desired properties, such as, for example, improved cellular uptake, improved stability, reduced immunogenicity, improved potency, improved target hybridization, susceptibility to RNAse cleavage, etc. Antisense nucleic acids can be modified at a base moiety, sugar moiety and/or phosphate backbone. Accordingly, antisense nucleic acids may have one or more modified nucleotides (e.g., a nucleotide analog) and/or one or more backbone modifications (e.g., a modified internucleotide linkage). Antisense nucleic acids may have a combination of modified and unmodified nucleotides. Antisense nucleic acids may also have a combination of modified and unmodified internucleotide linkages. Antisense nucleic acids may also consist entirely of modified nucleotides and/or modified internucleotide linkages.

Antisense nucleic acids may include ribonucleotides, deoxyribonucleotides, and combinations thereof. Examples of modified nucleotides which can be used in antisense nucleic acids include, for example, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, and 2,6-diaminopurine.

In some embodiments, a modified nucleotide is a 2′-modified nucleotide. For example, the 2′-modified nucleotide may be a 2′-deoxy, 2′-fluoro, 2′-O-methyl, 2′-O-methoxyethyl, 2′-amino and 2′-aminoalkoxy modified nucleotides. In some embodiments, the 2′-modified nucleotide comprises a 2′-O-4′-C methylene bridge, such as a locked nucleic acid (LNA) nucleotide. In some embodiments of a 2′ modified nucleotide the 2′-hydroxyl group is linked to the 3′ or 4′ carbon atom of the sugar ring thereby forming a bicyclic sugar moiety. In such embodiments, the linkage may be a methelyne (—CH2-)ₙ group bridging the 2′ oxygen atom and the 3′ or 4′ carbon atom wherein n is 1 or 2.

Antisense nucleic acids may include combinations of LNA nucleotides and unmodified nucleotides. Antisense nucleic acids may include combinations LNA and RNA nucleotides. Antisense nucleic acids may include combinations LNA and DNA nucleotides. A further preferred oligonucleotide modification includes Locked Nucleic Acids (LNAs) in which the 2′-hydroxyl group is linked to the 3′ or 4′ carbon atom of the sugar ring thereby forming a bicyclic sugar moiety.

Antisense nucleic acids may also include nucleobase-modified nucleotides, e.g., nucleotides containing a non-naturally occurring nucleobase instead of a naturally occurring nucleobase. Bases may be modified to block the activity of adenosine deaminase, for example. Examples of modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Within antisense nucleic acids (e.g., oligonucleotides) of the disclosures, as few as one and as many as all nucleotides can be modified. For example, an oligonucleotide (e.g., an oligonucleotide of 20 nucleotides in length) may contain 1, 2, 3, 4, 5, 6, 7, 8, 9, 10, 11, 12, 13, 14, 15, 16, 17, 18, 19 or 20 modified nucleotides. In some embodiments, a modified oligonucleotide will contain as few modified nucleotides as are necessary to achieve a desired level of in vivo stability and/or bioaccessibility or other desired property.

Certain antisense nucleic acids may include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain a diol, such as tetraethyleneglycol or hexaethyleneglycol, at either or both termini have also been shown to be substantially resistant to nuclease degradation and may be used herein. In some embodiments, antisense nucleic acids may include at least one lipophilic substituted nucleotide analog and/or a pyrimidine-purine dinucleotide.

In some embodiments, antisense nucleic acids (e.g., oligonucleotides) may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′-3′linkage may be a phosphodiester, phosphorothioate or any other modified internucleoside bridge. Additionally, 3′3′-linked oligonucleotides where the linkage between the 3′ terminal nucleosides is not a phosphodiester, phosphorothioate or other modified bridge, can be prepared using an additional spacer, such as tri- or tetra-ethylenglycol phosphate moiety.

A phosphodiester internucleotide linkage of an antisense nucleic acid can be replaced with a modified linkage. The modified linkage may be selected from, for example, phosphorothioate, phosphorodithioate, NR1R2-phosphoramidate, boranophosphate, α-hydroxybenzyl phosphonate, phosphate-(C1-C21)-O-alkyl ester, phosphate-[(C6-C12)aryl-(C1-C21)-O-alkyl]ester, (C1-C8)alkylphosphonate and/or (C6-C12)arylphosphonate bridges, and (C7-C12)-α-hydroxymethyl-aryl.

A phosphate backbone of the antisense nucleic acid can be modified to generate peptide nucleic acid molecules. As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols, for example.

Antisense nucleic acids can also be formulated as morpholino oligonucleotides. In such embodiments, the riboside moiety of each subunit of an oligonucleotide of the oligonucleotide reagent is converted to a morpholine moiety. Morpholinos may also be modified, e.g. as peptide conjugated morpholino).

In other embodiments, the antisense nucleic acid (e.g., oligonucleotide) can be linked to functional groups such as peptides (e.g., for targeting host cell receptors in vivo), or agents facilitating transport across the cell membrane or the blood-brain barrier. Oligonucleotide reagents of the disclosure also may be modified with chemical moieties (e.g., cholesterol) that improve the in vivo pharmacological properties of the oligonucleotide reagents.

Aspects of the disclosure relate to ASOs having a “gapmer” structure. As used herein, “gapmer” refers to a chimeric nucleic acid sequence comprising DNA bases and RNA bases arranged as follows: (modified RNA nucleobase)_{N}-(unmodified DNA nucleobase)_{A}-(modified RNA nucleobase)_{N}, where each “N” is an integer between 1 and 20 and “A” is an integer between 2 and 10. Examples of ASOs having a “gapmer” structure include: ASOs with a “5-10-5” structure containing 5 ribonucleotides (starting from the 5′ end) with 2′-O-methoxyethyl modifications, followed by 10 deoxynucleotides, followed by 5 ribonucleotides with 2′-O-methoxyethyl modifications, with all with phosphorothioate internucleotide linkages. In some embodiments, the cytidine nucleotides of a gapmer may be methylated. In some embodiments, the uridine nucleotides of a gapmer may be methylated. In some embodiments, the gapmer structure will include 15 nucleotides with alternating LNA type and deoxy-type nucleotides, all with phosphorothioate internucleotide linkages. In some embodiments, the central two nucleotides of a gapmer are deoxynucleotides. In some embodiments, an isolated nucleic acid having the sequence set forth in any one of SEQ ID NOs: 2-8, 10-24, 26-39, 41-50, 52-79, and 81-90 comprises a “gapmer” structure (the skilled artisan will recognize that in any of the nucleic acid sequences described herein, one or more “T” DNA nucleobases may be substituted with a “U” RNA nucleobase, or vice versa, in order to produce an ASO having a gapmer structure).

### Nucleic Acids Targeting AAV ITRs

Aspects of the disclosure relate to methods for modulating transgene expression (e.g., transgene expression mediated by an rAAV vector or expression cassette) by contacting a cell configured to express the transgene with one or more (e.g., 1, 2, 3, 4, 5, or more) isolated nucleic acids that bind to an AAV inverted terminal repeat (ITR) (e.g., a nucleic acid sequence encoding an AAV ITR). In some embodiments, binding of an isolated nucleic acid described herein to an AAV ITR sequence increases expression of the rAAV vector containing the ITR or increases transduction efficiency of the rAAV vector containing the ITR.

An isolated nucleic acid may specifically bind to a 5′ ITR, a 3′ ITR, or both 5′ and a 3′ ITRs of an rAAV vector. In some embodiments, an isolated nucleic acid binds (e.g., hybridizes) to an AAV2 ITR. In some embodiments, an AAV2 ITR comprises or consists of the sequence set forth in SEQ ID NO: 1. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of an AAV2 ITR (e.g., having the sequence as set forth in SEQ ID NO: 1). In some embodiments, the isolated nucleic acids comprise or consist of the nucleic acid sequence set forth in any one of SEQ ID NOs: 2-8.

The modulation (e.g., increase or decrease) in transgene expression caused by binding of the one or more isolated nucleic acids (e.g., ASOs) to an AAV ITR may vary. In some embodiments, binding of the one or more isolated nucleic acids (e.g., ASOs) to an AAV ITR results in an increase in transgene expression of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to an AAV ITR results in an increase in transduction efficiency of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to an AAV ITR results in a decrease in transgene expression of about 1%, 5%, 10%, 20%, 50%, 100%, 500%, or 1000%, relative to transgene expression of an rAAV vector that has not been contacted with the one or more isolated nucleic acids.

### Nucleic Acids Targeting Transcriptional Regulator Regions

Aspects of the disclosure relate to methods for modulating transgene expression (e.g., transgene expression mediated by an rAAV vector or expression cassette) by contacting a cell configured to express the transgene with one or more (e.g., 1, 2, 3, 4, 5, or more) isolated nucleic acids that bind to a transcriptional control region sequence (e.g., a nucleic acid sequence encoding one or more transcriptional regulators). Examples of transcriptional control region sequences include promoter sequences, enhancer sequences, repressor sequences, Kozak sequences, etc. In some embodiments, a transcriptional control region sequence comprises a promoter sequence. In some embodiments, a transcriptional control region sequence comprises an enhancer sequence. In some embodiments, binding of an isolated nucleic acid described herein to a transcriptional regulator sequence increases expression of the rAAV vector or decreases transduction efficiency of the rAAV vector.

An isolated nucleic acid may specifically bind to a promoter sequence of an rAAV vector. A promoter sequence may be a constitutive promoter sequence, inducible promoter sequence, tissue-specific promoter sequence, native promoter sequence, etc. In some embodiments, an isolated nucleic acid binds (e.g., hybridizes) to a chicken-beta actin (CBA) promoter sequence. In some embodiments, a CBA promoter sequence comprises or consists of the sequence set forth in SEQ ID NO: 9. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of a CBA promoter sequence (e.g., having the sequence as set forth in SEQ ID NO: 9). In some embodiments, the isolated nucleic acids comprise or consist of the nucleic acid sequence set forth in any one of SEQ ID NOs: 10-24.

An isolated nucleic acid may specifically bind to an enhancer sequence of an rAAV vector. In some embodiments, an isolated nucleic acid binds (e.g., hybridizes) to a cytomegalovirus (CMV) enhancer sequence. In some embodiments, a CMV enhancer sequence comprises or consists of the sequence set forth in SEQ ID NO: 25. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of a CMV promoter sequence (e.g., having the sequence as set forth in SEQ ID NO: 25). In some embodiments, the isolated nucleic acids comprise or consist of the nucleic acid sequence set forth in any one of SEQ ID NOs: 26-39.

The modulation (e.g., increase or decrease) in transgene expression caused by binding of the one or more isolated nucleic acids (e.g., ASOs) to a transcriptional control region sequence may vary. In some embodiments, binding of the one or more isolated nucleic acids (e.g., ASOs) to a transcriptional control region sequence results in an increase in transgene expression of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to a transcriptional control region sequence results in a decrease in transgene expression of about 1%, 5%, 10%, 20%, 50%, 100%, 500%, or 1000%, relative to transgene expression of an rAAV vector that has not been contacted with the one or more isolated nucleic acids.

### Nucleic Acids Targeting Coding Sequences

Aspects of the disclosure relate to methods for modulating transgene expression (e.g., transgene expression mediated by an rAAV vector or expression cassette) by contacting a cell configured to express the transgene with one or more (e.g., 1, 2, 3, 4, 5, or more) isolated nucleic acids that bind a protein coding region (e.g., a DNA or mRNA sequence) encoding one or more therapeutic genes (e.g., a PD-associated gene), for example a Gcase (e.g., the gene product of GBA1 gene) or a portion thereof, a progranulin (e.g., the gene product of PGRN gene) or portion thereof, a prosaposin (e.g., the gene product of PSAP gene) or portion thereof, a triggering receptor expressed on myeloid cells 2 (e.g., the gene product of TREM2 gene) or a portion thereof, an apolipoprotein (e.g., the gene product of APOE gene), a C9Orf72 protein (e.g., the gene product of C9Orf72 gene) or portion thereof, etc. In some embodiments, a gene product is encoded by a coding portion (e.g., a cDNA) of a naturally occurring gene. In some embodiments, a coding region encodes a protein fragment of a natural occurring gene. A protein fragment may comprise about 50%, about 60%, about 70%, about 80% about 90% or about 99% of a naturally occurring protein. In some embodiments, a protein fragment comprises between 50% and 99.9% (e.g., any value between 50% and 99.9%) of a naturally occurring protein. In some embodiments, binding of an isolated nucleic acid described herein to a transcriptional regulator sequence decreases expression of the protein from the rAAV vector.

An isolated nucleic acid may specifically bind to a protein coding region of an rAAV vector (e.g., an mRNA transcribed from an rAAV vector). In some embodiments, an isolated nucleic acid binds (e.g., hybridizes) to a protein coding region (e.g., a DNA or mRNA sequence) encoding any one of the foregoing transgenes, or a gene product thereof, or a codon-optimized region of the transgene, or a codon-optimized region of the gene product. In some embodiments, an isolated nucleic acid binds (e.g., hybridizes) to a protein coding region encoding any one of the foregoing transgenes, or a gene product thereof, or a codon-optimized region of the transgene, or a codon-optimized region of the gene product. In some embodiments, an isolated nucleic acid binds (e.g., hybridizes) to a protein coding region that encodes β-glucocerebrosidase or GBA. Gcase, also referred to as β-glucocerebrosidase or GBA, refers to a lysosomal protein that cleaves the beta-glucosidic linkage of the chemical glucocerebroside, an intermediate in glycolipid metabolism. In humans, Gcase is encoded by the GBA1 gene, located on chromosome 1. In some embodiments, GBA1 encodes a peptide that is represented by NCBI Reference Sequence NCBI Reference Sequence NP_000148.2.

In some embodiments, an isolated nucleic acid specifically binds to a codon optimized (e.g., codon optimized for expression in mammalian cells, for example human cells) nucleic acid sequence encoding GBA protein, such as the sequence set forth in SEQ ID NO: 40. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of a GBA protein coding sequence (e.g., having the sequence as set forth in SEQ ID NO: 40). In some embodiments, the isolated nucleic acids comprise or consist of the nucleic acid sequence set forth in any one of SEQ ID NOs: 41-50 or any one of SEQ ID NOs: 91-95.

In some embodiments, an isolated nucleic acid that specifically binds to a protein coding region (e.g., a nucleic acid sequence encoding GBA protein, such as an mRNA sequence encoding GBA protein) comprises a gapmer structure. In some embodiments, an isolated nucleic acid having a gapmer structure comprises at least 3 contiguous nucleotides of the sequence set forth in any one of SEQ ID NOs: 41-50, any one of SEQ ID NOs: 91-95, or any one of SEQ ID NOs: 106-110.

The modulation (e.g., increase or decrease) in transgene expression caused by binding of the one or more isolated nucleic acids (e.g., ASOs) to a protein coding sequence may vary. In some embodiments, binding of the one or more isolated nucleic acids (e.g., ASOs) to a transcriptional control region sequence results in an increase in transgene expression of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to a protein coding sequence results in a decrease in transgene expression of about 1%, 5%, 10%, 20%, 50%, 100%, 500%, or 1000%, relative to transgene expression of an rAAV vector that has not been contacted with the one or more isolated nucleic acids.

### Nucleic Acids Targeting Post-Transcriptional Regulatory Elements

Aspects of the disclosure relate to methods for modulating transgene expression (e.g., transgene expression mediated by an rAAV vector or expression cassette) by contacting a cell configured to express the transgene with one or more (e.g., 1, 2, 3, 4, 5, or more) isolated nucleic acids that bind to a post-transcriptional regulatory element sequence (e.g., a nucleic acid sequence such as an mRNA comprising a post-transcriptional regulatory element sequence). Examples of poly-adenylation element sequences include Hepatitis B virus (HPRE) and Woodchuck Hepatitis virus (WPRE), etc. In some embodiments, a post-transcriptional regulatory element sequence is a woodchuck post-transcriptional regulatory element sequence (WPRE). In some embodiments, binding of an isolated nucleic acid described herein to a post-transcriptional regulatory element sequence increases expression of the rAAV vector or decreases transduction efficiency of the rAAV vector.

An isolated nucleic acid may specifically bind to a WPRE of an rAAV vector. In some embodiments, a WPRE element sequence comprises or consists of the sequence set forth in SEQ ID NO: 51. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of a WPRE sequence (e.g., having the sequence as set forth in SEQ ID NO: 51). In some embodiments, the isolated nucleic acids comprise or consist of the nucleic acid sequence set forth in any one of SEQ ID NOs: 52-79, any one of SEQ ID NOs: 96-100, or any one of SEQ ID NOs: 111-115.

The modulation (e.g., increase or decrease) in transgene expression caused by binding of the one or more isolated nucleic acids (e.g., ASOs) to a post-transcriptional regulatory element sequence (e.g., WPRE) may vary. In some embodiments, binding of the one or more isolated nucleic acids (e.g., ASOs) to post-transcriptional regulatory element sequence results in an increase in transgene expression of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to a post-transcriptional regulatory element sequence results in a decrease in transgene expression of about 1%, 5%, 10%, 20%, 50%, 100%, 500%, or 1000%, relative to transgene expression of an rAAV vector that has not been contacted with the one or more isolated nucleic acids.

### Nucleic Acids Targeting Poly-Adenylation Elements

Aspects of the disclosure relate to methods for modulating transgene expression (e.g., transgene expression mediated by an rAAV vector or expression cassette) by contacting a cell configured to express the transgene with one or more (e.g., 1, 2, 3, 4, 5, or more) isolated nucleic acids that bind to a poly-adenylation element sequence (e.g., a nucleic acid sequence such as an DNA encoding a poly-A tail or mRNA comprising a poly-U tail). Examples of poly-adenylation element sequences include SV40 poly-adenylation element, bovine growth hormone (BGH) poly-adenylation element, etc. In some embodiments, a poly-adenylation element is a BGH poly-A element. In some embodiments, binding of an isolated nucleic acid described herein to a transcriptional regulator sequence increases expression of the rAAV vector or decreases transduction efficiency of the rAAV vector.

An isolated nucleic acid may specifically bind to a BGH poly-A element of an rAAV vector. In some embodiments, a BGH poly-adenylation element sequence comprises or consists of the sequence set forth in SEQ ID NO: 80. In some embodiments, an isolated nucleic acid specifically binds to (e.g., hybridizes with) at least 2, 3, 4, 5, 6, 7, 8, 9, 10, 11, 12, 13, 14, 15, 16, 17, 18, 19, 20, 21, 22, 23, 24, or 25 nucleotides of a BGH poly-adenylation element sequence (e.g., having the sequence as set forth in SEQ ID NO: 80). In some embodiments, the isolated nucleic acids comprise or consist of the nucleic acid sequence set forth in any one of SEQ ID NOs: 81-90 or SEQ ID NOs: 101-104.

In some embodiments, an isolated nucleic acid that specifically binds to a poly-adenylation element sequence (e.g., a nucleic acid sequence encoding BGH poly-adenylation element, such as an mRNA sequence encoding a BGH poly-adenylation element) comprises a gapmer structure. In some embodiments, an isolated nucleic acid having a gapmer structure comprises at least 3 contiguous nucleotides of the sequence set forth in any one of SEQ ID NOs: 81-90, any one of SEQ ID NOs: 101-104, or any one of SEQ ID NOs: 116-120.

The modulation (e.g., increase or decrease) in transgene expression caused by binding of the one or more isolated nucleic acids (e.g., ASOs) to a BGH poly-adenylation element sequence may vary. In some embodiments, binding of the one or more isolated nucleic acids (e.g., ASOs) to a BGH poly-adenylation element sequence results in an increase in transgene expression of about 1%, 5%, 10%, 20%, 50%, 100%, 500%, 1000% or more, relative to transgene expression of an rAAV vector that has not been contacted with the one or more isolated nucleic acids. In some embodiments, binding of the one or more isolated nucleic acids (e.g., ASOs) to a BGH poly-adenylation element sequence results in a decrease in transgene expression of about 1%, 5%, 10%, 20%, 50%, 100%, 500%, or 1000%, relative to transgene expression of an rAAV vector that has not been contacted with the one or more isolated nucleic acids.

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference. Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

rAAV Vectors and rAAVs

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, an rAAV vector is single-stranded (e.g., single-stranded DNA). In some embodiments, the vector is a recombinant AAV (rAAV) vector. In some embodiments, a vector is a Baculovirus vector (e.g., an Autographa californica nuclear polyhedrosis (AcNPV) vector).

Typically an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26):2112-8.

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived. In some embodiments, an AAV capsid protein variant is an AAV1RX capsid protein, for example as described by Albright et al. Mol Ther. 2018 Feb. 7; 26(2):510-523. In some embodiments, a capsid protein variant is an AAV TM6 capsid protein, for example as described by Rosario et al. Mol Ther Methods Clin Dev. 2016; 3: 16026.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63(9):3822-8 and Wright (2009) Hum Gene Ther. 20(7): 698-706. In some embodiments, an rAAV comprises a capsid protein that specifically or preferentially targets myeloid cells, for example microglial cells.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes).

### Modulation of Gene Expression

Aspects of the disclosure relate to compositions and methods for positively or negatively regulating the expression of a gene therapeutic (e.g., a therapeutic protein expressed from an AAV vector), through the use of certain nucleic acids, for example antisense oligonucleotides (ASOs) that specifically bind (e.g., hybridize) to one or more of the following: viral vector regions (e.g., AAV ITRs), DNA or RNA regulatory elements (e.g., promoter sequences, enhancer sequences, post-transcriptional regulatory element sequences, etc.), and protein coding sequences of mRNA transcribed from rAAV vectors. An isolated nucleic acid may be administered to a cell or subject at the same time as the expression cassette or rAAV vector to which it specifically binds, or at a different time (e.g., before or after administration of the expression cassette or rAAV vector). In some embodiments, a subject is administered an rAAV and then subsequently administered one or more doses of an isolated nucleic acid (or isolated nucleic acids) as described herein. In some embodiments, a subject is administered one or more isolated nucleic acids based upon detecting a level of transgene expression in the cell or subject prior to the administration of the isolated nucleic acids.

In some embodiments, an rAAV vector is in a cell, such as a host cell. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell. In some embodiments, a cell is in vitro. In some embodiments, a cell is in a subject, for example a mammalian subject such as a human, mouse, dog, cat, etc.

In some embodiments, one isolated nucleic acid that specifically binds to an rAAV vector or expression cassette is provided to a cell or subject. In some embodiments, more than one (e.g., 2, 3, 4, 5, or more) isolated nucleic acids are provided to the cell or subject. The more than one different isolated nucleic acids (e.g., ASOs) may bind to the same region or sequence (e.g., two ASOs that each bind to an AAV ITR), or each to different regions or sequences (e.g., a first ASO that specifically binds to an AAV ITR and a second ASO that binds to a post-transcriptional regulatory element sequence).

Delivery of the one or more isolated nucleic acids described by the disclosure to a cell or subject, in some embodiments, results in modulation (e.g., increase or decrease) in transgene expression caused by binding of the one or more isolated nucleic acids (e.g., ASOs) to a sequence of an rAAV vector or expression construct contained in the cell or subject. In some embodiments, binding of the one or more isolated nucleic acids (e.g., ASOs) to an rAAV vector or expression construct results in an increase in transgene expression of about 1%, 5%, 10%, 20%, 50%, 100%, 500%, 100% or more, relative to transgene expression of an rAAV vector or expression construct that has not been contacted with the one or more isolated nucleic acids. In some embodiments, binding of the one or more isolated nucleic acids (e.g., ASOs) to an rAAV vector or expression construct results in an increase in transduction efficiency of about 1%, 5%, 10%, 20%, 50%, 100%, 500%, 100% or more, relative to transgene expression of an rAAV vector or expression construct that has not been contacted with the one or more isolated nucleic acids. In some embodiments, binding of the one or more isolated nucleic acids (e.g., ASOs) to an rAAV vector or expression construct results in a decrease in transgene expression of about 1%, 5%, 10%, 20%, 50%, 100%, 500%, or 100%, relative to transgene expression of an rAAV vector or expression construct that has not been contacted with the one or more isolated nucleic acids. In some embodiments, the one or more isolated nucleic acid is delivered to the cell at the same time as the cell is transfected with the plasmid comprising the rAAV vector encoding the transgene. In some embodiments, the one or more isolated nucleic acid is delivered to the cell after the cell is transfected with the plasmid comprising the rAAV vector encoding the transgene. In some embodiments, the one or more isolated nucleic acid is delivered to the cell 1, 2, 3, 4, 5, 6, 7, 8, 9, 10, 12, 14, 16, 18, 20, 22 or 24 hours after the cell is transfected with the plasmid comprising the rAAV vector encoding the transgene. In some embodiments, the one or more isolated nucleic acid is delivered to the cell 1, 2, 3, 4, 5, 6, 7, 8, 9, 10, 11, 12, 13, 14, 15, 16, 17, 18, 19 or 20 days after the cell is transfected with the plasmid comprising the rAAV vector encoding the transgene. In some embodiments, the one or more isolated nucleic acid is delivered to the cell 1, 2, 3, 4, 10, 15, 20, 26 or 52 weeks after the cell is transfected with the plasmid comprising the rAAV vector encoding the transgene. In some embodiments, the one or more isolated nucleic acid is delivered to the cell 1, 2, 3 or 5 years after the cell is transfected with the plasmid comprising the rAAV vector encoding the transgene.

Aspects of the disclosure relate to compositions for modulation of expression of one or more CNS disease-associated gene products in a subject to treat CNS-associated diseases. The one or more CNS disease-associated gene products may be encoded by one or more isolated nucleic acids or rAAV vectors. In some embodiments, a subject is administered a single vector (e.g., isolated nucleic acid, rAAV, etc.) encoding one or more (1, 2, 3, 4, 5, or more) gene products. In some embodiments, a subject is administered a plurality (e.g., 2, 3, 4, 5, or more) vectors (e.g., isolated nucleic acids, rAAVs, etc.), where each vector encodes a different CNS disease-associated gene product.

A CNS-associated disease may be a neurodegenerative disease, synucleinopathy, tauopathy, or a lysosomal storage disease. Examples of neurodegenerative diseases and their associated genes are listed in Table 1.

A “synucleinopathy” refers to a disease or disorder characterized by reduced expression or activity of alpha-Synuclein (the gene product of SCNA) in a subject (e.g., relative to a healthy subject, for example a subject not having a synucleinopathy). Examples of synucleinopathies and their associated genes are listed in Table 2.

A “tauopathy” refers to a disease or disorder characterized by reduced expression or activity of Tau protein in a subject (e.g., a healthy subject not having a tauopathy). Examples of tauopathies and their associated genes are listed in Table 3.

A “lysosomal storage disease” refers to a disease characterized by abnormal build-up of toxic cellular products in lysosomes of a subject. Examples of lysosomal storage diseases and their associated genes are listed in Table 4.

TABLE 1
Examples of neurodegenerative diseases

| Disease | Associated genes |
|---|---|
| Alzheimer's disease | APP, PSEN1, PSEN2, APOE |
| Parkinson's disease | LRRK2, PARK7, PINK1, PRKN, SNCA, GBA, UCHL1, |
| | ATP13A2, VPS35 |
| Huntington's disease | HTT |
| Amyotrophic lateral sclerosis | ALS2, ANG, ATXN2, C9orf72, CHCHD10, CHMP2B, |
| | DCTN1, ERBB4, FIG4, FUS, HNRNPA1, MATR3, |
| | NEFH, OPTN, PFN1, PRPH, SETX, SIGMAR1, |
| | SMN1, SOD1, SPG11, SQSTM1, TARDBP, TBK1, |
| | TRPM7, TUBA4A, UBQLN2, VAPB, VCP |
| Batten disease | PPT1, TPP1, CLN3, CLN5, CLN6, MFSD8, CLN8, |
| (Neuronal ceroid lipofunscinosis) | CTSD, DNAJC5, CTSF, ATP13A2, GRN, KCTD7 |
| Friedreich's ataxia | FXN |
| Lewy body disease | APOE, GBA, SNCA, SNCB |
| Spinal muscular atrophy | SMN1, SMN2 |
| Multiple sclerosis | CYP27B1, HLA-DRB1, IL2RA, IL7R, TNFRSF1A |
| Prion disease (Creutzfeldt- | PRNP |
| Jakob disease, Fatal familial | |
| insomnia, Gertsmann-Straussler- | |
| Scheinker syndrome, Variably | |
| protease-sensitive | |
| prionopathy) | |

TABLE 2
Examples of synucleinopathies

| Disease | Associated genes |
|---|---|
| Parkinson's disease | LRRK2, PARK7, PINK1, PRKN, |
| | SNCA, GBA, UCHL1, ATP13A2, VPS35 |
| Dementia with Lewy bodies | APOE, GBA, SNCA, SNCB |
| Multiple system atrophy | COQ2, SNCA |

TABLE 3
Examples of tauopathies

| Disease | Associated genes |
|---|---|
| Alzheimer's disease | APP, PSEN1, PSEN2, APOE |
| Primary age-related tauopathy | MAPT |
| Progressive supranuclear palsy | MAPT |
| Corticobasal degeneration | MAPT, GRN, C9orf72, VCP, |
| | CHMP2B, TARDBP, FUS |
| Frontotemporal dementia | MAPT |
| with parkinsonism-17 | |
| Subacute sclerosing panencephalitis | SCN1A |
| Lytico-Bodig disease | |
| Gangioglioma, gangliocytoma | |
| Meningioangiomatosis | |
| Postencephalitic parkinsonism | |
| Chronic traumatic encephalopathy | |

TABLE 4
Examples of lysosomal storage diseases

| Disease | Associated genes |
|---|---|
| Niemann-Pick disease | NPC1, NPC2, SMPD1 |
| Fabry disease | GLA |
| Krabbe disease | GALC |
| Gaucher disease | GBA |
| Tach-Sachs disease | HEXA |
| Metachromatic leukodystrophy | ARSA, PSAP |
| Farber disease | ASAH1 |
| Galactosialidosis | CTSA |
| Schindler disease | NAGA |
| GM1 gangliosidosis | GLB1 |
| GM2 gangliosidosis | GM2A |
| Sandhoff disease | HEXB |
| Lysosomal acid lipase deficiency | LIPA |
| Multiple sulfatase deficiency | SUMF1 |
| Mucopolysaccharidosis Type I | IDUA |
| Mucopolysaccharidosis Type II | IDS |
| Mucopolysaccharidosis Type III | GNS, HGSNAT, NAGLU, SGSH |
| Mucopolysaccharidosis Type IV | GALNS, GLB1 |
| Mucopolysaccharidosis Type VI | ARSB |
| Mucopolysaccharidosis Type VII | GUSB |
| Mucopolysaccharidosis Type IX | HYAL1 |
| Mucolipidosis Type II | GNPTAB |
| Mucolipidosis Type III alpha/beta | GNPTAB |
| Mucolipidosis Type III gamma | GNPTG |
| Mucolipidosis Type IV | MCOLN1 |
| Neuronal ceroid lipofuscinosis | PPT1, TPP1, CLN3, CLN5, |
| | CLN6, MFSD8, CLN8, CTSD, |
| | DNAJC5, CTSF, ATP13A2, |
| | GRN, KCTD7 |
| Alpha-mannosidosis | MAN2B1 |
| Beta-mannosidosis | MANBA |
| Aspartylglucosaminuria | AGA |
| Fucosidosis | FUCA1 |

As used herein “treat” or “treating” refers to (a) preventing or delaying onset of a CNS disease; (b) reducing severity of a CNS disease; (c) reducing or preventing development of symptoms characteristic of a CNS disease; and/or (d) preventing worsening of symptoms characteristic of a CNS disease in a subject. Symptoms of CNS disease may include, for example, motor dysfunction (e.g., shaking, rigidity, slowness of movement, difficulty with walking, paralysis), cognitive dysfunction (e.g., dementia, depression, anxiety, psychosis), difficulty with memory, emotional and behavioral dysfunction.

A subject is typically a mammal, preferably a human. In some embodiments, a subject is between the ages of 1 month old and 10 years old (e.g., 1 month, 2 months, 3 months, 4, months, months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3, years, 4 years, 5 years, 6 years, 7 years, 8 years, 9 years, 10 years, or any age therebetween). In some embodiments, a subject is between 2 years old and 20 years old. In some embodiments, a subject is between 30 years old and 100 years old. In some embodiments, a subject is older than 55 years old.

In some embodiments, one or more compositions is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV, and/or an isolated nucleic acid as described herein) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

In some embodiments, a subject is administered an immunosuppressant prior to (e.g., between 1 month and 1 minute prior to) or at the same time as a composition as described herein. In some embodiments, the immunosuppressant is a corticosteroid (e.g., prednisone, budesonide, etc.), an mTOR inhibitor (e.g., sirolimus, everolimus, etc.), an antibody (e.g., adalimumab, etanercept, natalizumab, etc.), or methotrexate.

The amount of composition (e.g., a composition comprising an isolated nucleic acid or a vector or a rAAV) as described by the disclosure administered to a subject will vary depending on the administration method.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Examples 1-5: Cell Based Assays of Viral Transduction into GBA-Deficient Cells

Cells deficient in GBA1 are obtained, for example as fibroblasts from GD patients, monocytes, or hES cells, or patient-derived induced pluripotent stem cells (iPSCs), or HEK293T cells. These cells accumulate substrates such as glucosylceramide and glucosylsphingosine (GlcCer and GlcSph). Treatment of wild-type or mutant cultured cell lines with Gcase inhibitors, such as CBE, is also be used to obtain GBA deficient cells.

The cells are administered an rAAV comprising an AAV9 capsid protein enclosing an rAAV vector comprising the nucleic acid sequences set forth in SEQ ID NOs: 1, 9, 25, 40, 51, and 80 (e.g., PR001). Transduction efficiency and GBA expression level is monitored in the cells. One or more isolated nucleic acids (e.g., ASOs) targeting the PR001 vector are administered to the cell. Therapeutic endpoints (e.g., reduction of PD-associated pathology for in vivo assays) are measured in the context of expression of transduction of the AAV vectors, to confirm and quantify increase or decrease in activity and function of PR001. GBA expression is quantified using qRT-PCT, or through Gcase level measurement using protein ELISA measures, or by standard Gcase activity assays.

### Example 1: Effect of ASOs Directed Against GBA

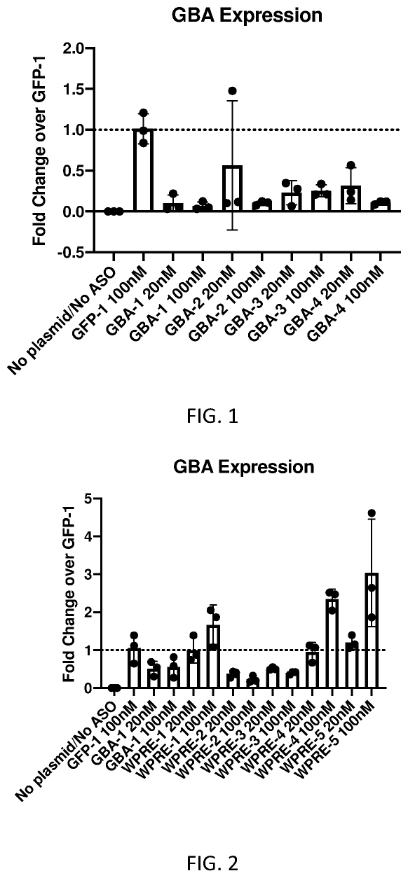

HEK293T cells were transfected with a plasmid comprising an rAAV vector encoding GBA protein (e.g., PR001) and administered ASOs directed against the GBA-encoding portion of the rAAV vector (PR001). The levels and types of ASOs administered in the eight experimental groups were as follows: 20 nM GBA ASO 1 modified (SEQ ID NO: 91), 100 nM GBA ASO 1 modified (SEQ ID NO: 91), 20 nM GBA ASO 2 modified (SEQ ID NO: 92), 100 nM GBA ASO 2 modified (SEQ ID NO: 92), 20 nM GBA ASO 3 modified (SEQ ID NO: 93), 100 nM GBA ASO 3 modified (SEQ ID NO: 93), 20 nM GBA ASO 4 modified (SEQ ID NO: 94), 100 nM GBA ASO 4 modified (SEQ ID NO: 94). A negative control group and a positive control group were also included in the experimental design. In the negative control, cells were not transfected by the GBA-encoding plasmid and were not administered any ASOs. In the positive control, cells were transfected with the plasmid encoding GBA expression and administered 100 nM of an ASO directed against GFP (Green Fluorescent Protein), comprising the nucleic acid sequence set forth in SEQ ID NO: 105. Cells were harvested after 72 hours and GBA expression was quantified using qRT-PCR. All experimental groups showed a significant decrease in GBA expression compared to the positive control (FIG. 1).

### Example 2: Effect of ASOs Directed Against WPRE

HEK293T cells were transfected with a plasmid comprising an rAAV vector encoding GBA protein (e.g., PR001) and administered ASOs directed against the WPRE-encoding portion of the rAAV vector (PR001). The levels and types of ASOs administered in the ten experimental groups were as follows: 20 nM WPRE ASO 1 modified (SEQ ID NO: 96), 100 nM WPRE ASO 1 modified (SEQ ID NO: 96), 20 nM WPRE ASO 2 modified (SEQ ID NO: 97), 100 nM WPRE ASO 2 modified (SEQ ID NO: 97), 20 nM WPRE ASO 3 modified (SEQ ID NO: 98), 100 nM WPRE ASO 3 modified (SEQ ID NO: 98), 20 nM WPRE ASO 4 modified (SEQ ID NO: 99), 100 nM WPRE ASO 4 modified (SEQ ID NO: 99), 20 nM WPRE ASO 5 modified (SEQ ID NO: 100), 100 nM WPRE ASO 5 modified (SEQ ID NO: 100). Four control groups were also included in the experimental design: a negative control group where cells were not transfected by the GBA-encoding plasmid and were not administered any ASOs, a positive control group where cells were transfected with the plasmid encoding GBA expression and administered 100 nM of an ASO directed against GFP (SEQ ID NO: 105), and two groups where cells were transfected with the GBA-encoding plasmid and administered 20 nM GBA ASO 1 modified (SEQ ID NO: 91) and 100 nM GBA ASO 1 modified (SEQ ID NO: 91) respectively. Cells were harvested after 72 hours and GBA expression was quantified using qRT-PCR. Surprisingly, increased GBA expression compared to the positive control was observed in several of the experimental groups, notably the groups that were administered 100 nM of WPRE ASO 1 modified (SEQ ID NO: 96), WPRE ASO 4 modified (SEQ ID NO: 99), or WPRE ASO 5 modified (SEQ ID NO: 100) (FIG. 2).

### Example 3: Effect of ASOs Directed Against the Bovine Growth Hormone Poly-Adenylation Element (BGH PolyA)

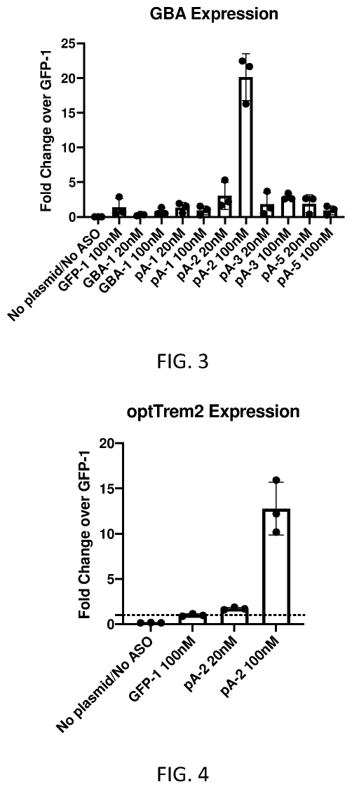

HEK293T cells were transfected with a plasmid comprising an rAAV vector encoding GBA protein (e.g., PR001) and administered ASOs directed against the BGH poly-adenylation element (PolyA)-encoding portion of the rAAV vector (PR001). The levels and types of ASOs administered in the eight experimental groups were as follows: 20 nM PolyA ASO 1 modified (SEQ ID NO: 101), 100 nM PolyA ASO 1 modified (SEQ ID NO: 101), 20 nM PolyA ASO 2 modified (SEQ ID NO: 102), 100 nM PolyA ASO 2 modified (SEQ ID NO: 102), 20 nM PolyA ASO 3 modified (SEQ ID NO: 103), 100 nM PolyA ASO 3 modified (SEQ ID NO: 103), 20 nM PolyA ASO 5 modified (SEQ ID NO: 104), 100 nM PolyA ASO 5 modified (SEQ ID NO: 104). Four control groups were also included in the experimental design: a negative control group where cells were not transfected by the GBA-encoding plasmid and were not administered any ASOs, a positive control group where cells were transfected with the plasmid encoding GBA expression and administered 100 nM of an ASO directed against GFP (SEQ ID NO: 105), and two groups where cells were transfected with the GBA-encoding plasmid and administered nM GBA ASO 1 modified (SEQ ID NO: 91) or 100 nM GBA ASO 1 modified (SEQ ID NO: 91) respectively. Cells were harvested after 72 hours and GBA expression was quantified using qRT-PCR. Surprisingly, all experimental groups showed similar or significantly increased GBA expression compared to the positive control. Notably, a 20-fold increase in GBA expression compared to the positive control was observed in the cells that were administered 100 nM of PolyA ASO 2 modified (SEQ ID NO: 102) (FIG. 3).

### Example 4: Effect of PolyA ASO 2 Modified (SEQ ID NO: 102) on Trem2 Expression

HEK293T cells were transfected with a plasmid comprising an rAAV vector encoding Trem2 protein and administered 20 nM or 100 nM of PolyA ASO 2 modified (SEQ ID NO: 102), respectively. Two control groups were also included in the experimental design: a negative control group where cells were not transfected by the Trem2-encoding plasmid and were not administered any ASOs, and a positive control group where cells were transfected with the plasmid encoding Trem2 expression and administered 100 nM of an ASO directed against GFP (SEQ ID NO: 105). Cells were harvested after 72 hours and Trem2 expression was quantified using qRT-PCR. Both experimental groups showed a significant increase in Trem2 expression compared to the positive control. Notably, the cells administered 100 nM of PolyA ASO 2 2 modified (SEQ ID NO: 102) showed a 13-fold increase in Trem2 expression compared to the positive control (FIG. 4).

### Example 5: Effect of Administering ASOs in Sequential Transfection

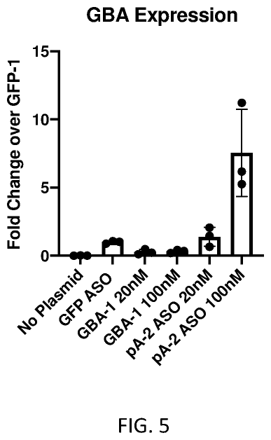

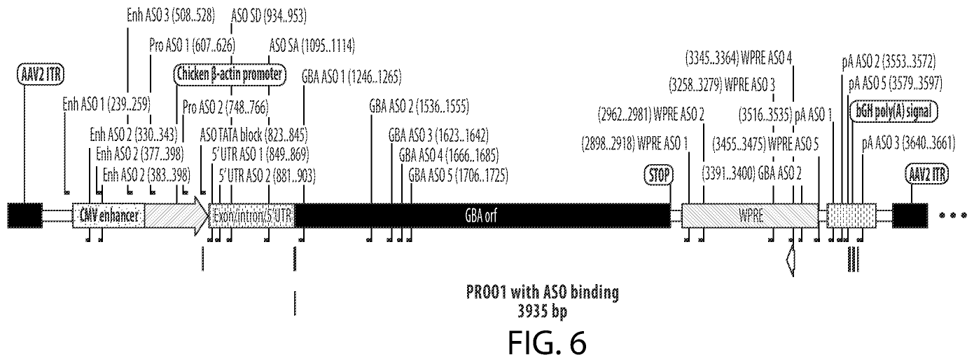

HEK293T cells were transfected with a plasmid comprising an rAAV vector encoding GBA protein. After 3 hours, the plasmid transfection mixture was removed and cells in four experimental groups were transfected with: 20 nM GBA ASO 1 modified (SEQ ID NO: 91), 100 nM GBA ASO 1 modified (SEQ ID NO: 91), 20 nM of PolyA ASO 2 modified (SEQ ID NO: 102) or 100 nM of PolyA ASO 2 modified (SEQ ID NO: 102), respectively. Two control groups were also included in the experimental design: a negative control group where cells were not transfected by the GBA-encoding plasmid and were not administered any ASOs, and a positive control group where cells were transfected with the plasmid encoding GBA expression and administered 100 nM of an ASO directed against GFP (SEQ ID NO: 105). Cells were harvested after 72 hours and GBA expression was quantified using qRT-PCR. Both experimental groups that were administered GBA ASO 1 modified (SEQ ID NO: 91) showed a significant decrease in GBA expression compared to the positive control. The group that was administered 20 nM of PolyA ASO 2 modified (SEQ ID NO: 102) showed similar levels of GBA expression as the positive group. The cells that were administered 100 nM of PolyA ASO 2 modified (SEQ ID NO: 102) had the most significant change, with a 7-8 fold increase in GBA expression compared to the positive control (FIG. 5).

### Example 6: In Vivo Assays

C57/BL6J male mice received IV infusions of an AAV expressing GBA (AAV-GBA) at 1×10¹² vg/kg or excipient control. Mice in the excipient group then received saline control, while mice received AAV-GBA then received IV infusions of saline or an ASO 7, 14 and 21 days following AAV-GBA infusion, as detailed in Table 5. Plasma was collected by submandibular bleed before AAV-GBA infusion and 7- and 21-days post AAV-GBA infusion. Mice were euthanized and tissues were collected for analysis 30 days after AAV infusion.

TABLE 5
In vivo assays

| Group | Virus | ASO | ASO dose | N |
|---|---|---|---|---|
| 1 | Excipient | None | n/a | 8 |
| 2 | AAV-GBA | None | n/a | 8 |
| 3 | AAV-GBA | GBA ASO 1 | 20 mg/kg* | 7 |
| | | (SEQ ID NO: 91) | | |
| 4 | AAV-GBA | WPRE ASO 2 | 50 mg/kg | 8 |
| | | (SEQ ID NO: 97) | | |
| 5 | AAV-GBA | PolyA ASO 2 | 5 mg/kg* | 5 |
| | | (SEQ ID NO: 102) | | |

*3 mice from group 3 and 1 mouse from group 5 had a day 7 ASO dose of 50 mg/kg

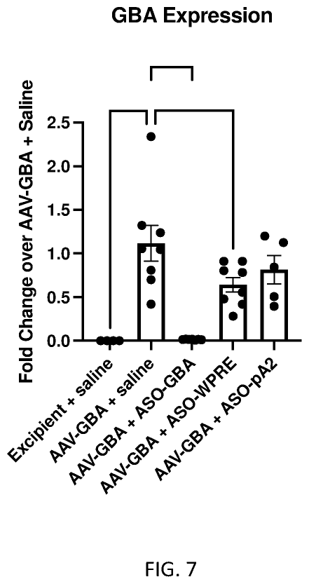

The results show that GBA ASO 1 and WPRE ASO 2 significantly decreased GBA mRNA level in the liver, and PolyA ASO 2 showed a trend in decreasing GBA mRNA in the liver (FIG. 7).

In vivo assays of AAV vectors are performed using mutant mice. The intrathecal or intraventricular delivery of vehicle control and AAV vectors (e.g., PR001 at a dose of 2×10¹¹ vg/mouse) are performed using concentrated AAV stocks, for example at an injection volume between 5-10 μL. Intraparenchymal delivery by convection enhanced delivery is performed. One or more isolated nucleic acids (e.g., ASOs) targeting the PR001 vector are administered to the cell. Therapeutic endpoints (e.g., reduction of PD-associated pathology) are measured in the context of expression of transduction of the AAV vectors, to confirm and quantify increase or decrease in activity and function of PR001. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### SEQUENCES

In some embodiments, an isolated nucleic acid described herein (e.g., an ASO) specifically binds (e.g., hybridizes) to a sequence set forth below, or the complement thereof, or the reverse complement thereof. In some embodiments, an isolated nucleic acid described herein (e.g., an ASO) comprises or consists of one of the sequences set forth below, or the complement thereof, or the reverse complement thereof, or a gapmer thereof, or a modified version thereof that comprises one or more chemical modifications, wherein the one or more chemical modification is selected from a nucleobase modification or a backbone modification. In some embodiments, all the nucleobases and/or the entire backbone of the ASO are modified. In some embodiments, a nucleobase modification comprises a 2′-O-methyl (2′OMe) modification, and a backbone modification comprises a phosphorothioate linkage. In some embodiments, an ASO comprises one or more locked nucleic acids (LNAs). In the sequences set forth below, the letter “m” preceding a nucleobase letter indicates a 2′-O-methyl (2′OMe) modification, and a “*” between two nucleobases indicates a phosphorothioate linkage.

>AAV2 ITR nucleic acid sequence
(SEQ ID NO: 1)
cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttcct
>ITR ASO 1
(SEQ ID NO: 2)
gcgcgcagctgcctgcagg
>ITR ASO 2
(SEQ ID NO: 3)
cggcctcagtgagcgagcga
>ITR ASO 3
(SEQ ID NO: 4)
acgcccgggctttgcccggg
>ITR ASO 4
(SEQ ID NO: 5)
cgggcgaccaaaggtcgcccg
>ITR ASO 5
(SEQ ID NO: 6)
gctcgctcgctcactgaggc
>ITR ASO 6
(SEQ ID NO: 7)
tggccactccctctctgcgc
>ITR ASO 7
(SEQ ID NO: 8)
aggaacccctagtgatggagt
>CMV Enhancer nucleic acid sequence
(SEQ ID NO: 9)
cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catg
>CMV ASO 1
(SEQ ID NO: 10)
tttaccgtaagttatgtaacg
>CMV ASO 2
(SEQ ID NO: 11)
ggcggtcagccaggcgggcca
>CMV ASO 3
(SEQ ID NO: 12)
gtcaatgggcgggggtcgttg
>CMV ASO 4
(SEQ ID NO: 13)
ggaacatacgtcattattgac
>CMV ASO 5
(SEQ ID NO: 14)
gtccctattggcgttactatg
>CMV ASO 6
(SEQ ID NO: 15)
acccattgacgtcaatggaaa
>CMV ASO 7
(SEQ ID NO: 16)
gcagtttaccgtaaatactcc
>CMV ASO 8
(SEQ ID NO: 17)
acttgatgtactgccaagtgg
>CMV ASO 9
(SEQ ID NO: 18)
ggcgtacttggcatatgatac
>CMV ASO 10
(SEQ ID NO: 19)
ccgtcattgacgtcaataggg
>CMV ASO 11
(SEQ ID NO: 20)
taatgccaggcgggccattta
>CMV ASO 12
(SEQ ID NO: 21)
cataaggtcatgtactgggca
>CMV ASO 13
(SEQ ID NO: 22)
tactgccaagtaggaaagtcc
>CMV ASO 14
(SEQ ID NO: 23)
gcgatgactaatacgtagatg
>CMV ASO 15
(SEQ ID NO: 24)
ccatggtaatagcgatgac
>CBA promoter nucleic acid sequence
(SEQ ID NO: 25)
tcgaggtgagccccacgttctgcttcactctccccatctcccccccctcc
ccacccccaattttgtatttatttattttttaattattttgtgcagcgat
gggggcggggggggggggggggcgcgcgccaggcggggcggggcggggcg
aggggcggggcggggcgaggcggagaggtgcggcggcagccaatcagagc
ggcgcgctccgaaagtttccttttatggcgaggcggcggcggcggcggcc
ctataaaaagcgaagcgcgcggcgggcg
>CB ASO 1
(SEQ ID NO: 26)
agaacgtggggctcacctcga
>CB ASO 2
(SEQ ID NO: 27)
gggagatggggagagtgaagc
>CB ASO 3
(SEQ ID NO: 28)
aaattgggggtggggaggggg
>CB ASO 4
(SEQ ID NO: 29)
attaaaaaataaataaataca
>CB ASO 5
(SEQ ID NO: 30)
cccccatcgctgcacaaaata
>CB ASO 6
(SEQ ID NO: 31)
cgcgccccccccccccccccg
>CB ASO 7
(SEQ ID NO: 32)
cccgccccgccccgcctggcg
>CB ASO 8
(SEQ ID NO: 33)
tcgccccgccccgcccctcgc
>CB ASO 9
(SEQ ID NO: 34)
ctgccgccgcacctctccgcc
>CB ASO 10
(SEQ ID NO: 35)
ggagcgcgccgctctgattgg
>CB ASO 11
(SEQ ID NO: 36)
tcgccataaaaggaaactttc
>CB ASO 12
(SEQ ID NO: 37)
agggccgccgccgccgccgcc
>CB ASO 13
(SEQ ID NO: 38)
gccgcgcgcttcgctttttat
>CB ASO 14
(SEQ ID NO: 39)
agcgcgcagcgactcccgccc
>GBA codon-optimized nucleic acid sequence
(SEQ ID NO: 40)
auggaauucagcagccccagcagagaggaaugccccaagccucugagccg
ggugucaaucauggccggaucucugacaggacugcugcugcuucaggccg
ugucuugggcuucuggcgcuagaccuugcauccccaagagcuucggcuac
agcagcgucgugugcgugugcaaugccaccuacugcgacagcuucgaccc
uccuaccuuuccugcucugggcaccuucagcagauacgagagcaccagau
ccggcagacggauggaacugagcaugggacccauccaggccaaucacaca
ggcacuggccugcugcugacacugcagccugagcagaaauuccagaaagu
gaaaggcuucggcggagccaugacagaugccgccgcucugaauauccugg
cucugucuccaccagcucagaaccugcugcucaagagcuacuucagcgag
gaaggcaucggcuacaacaucaucagagugcccauggccagcugcgacuu
cagcaucaggaccuacaccuacgccgacacacccgacgauuuccagcugc
acaacuucagccugccugaagaggacaccaagcugaagaucccucugauc
cacagagcccugcagcuggcacaaagacccgugucacugcuggccucucc
auggacaucucccaccuggcugaaaacaaauggcgccgugaauggcaagg
gcagccugaaaggccaaccuggcgacaucuaccaccagaccugggccaga
uacuucgugaaguuccuggacgccuaugccgagcacaagcugcaguuuug
ggccgugacagccgagaacgaaccuucugcuggacugcugagcggcuacc
ccuuucagugccugggcuuuacacccgagcaccaggggacuuuaucgccc
gugaucugggacccacacuggccaauagcacccaccauaaugugcggcug
cugaugcuggacgaccagagacugcuucugccccacugggcuaaaguggu
gcugacagauccugaggccgccaaauacgugcacggaaucgccgugcacu
gguaucuggacuuucuggccccugccaaggccacacugggagagacacac
agacuguuccccaacaccaugcuguucgccagcgaagccugugugggcag
caaguuuugggaacagagcgugcggcucggcagcugggauagaggcaugc
aguacagccacagcaucaucaccaaccugcuguaccacgucgucggcugg
accgacuggaaucuggcccugaauccugaaggggcccuaacuggguccga
aacuucguggacagccccaucaucguggacaucaccaaggacaccuucua
caagcagcccauguucuaccaccugggacacuucagcaaguucauccccg
agggcucucagcgcguuggacugguggcuucccagaagaacgaucuggac
gccguggcucugaugcacccugauggaucugcugugguggugguccugaa
ccgcagcagcaaagaugugccccugaccaucaaggaucccgccgugggau
uccuggaaacaaucagcccuggcuacuccauccacaccuaccuguggcgu
agacaguga
>GBA ASO 1
(SEQ ID NO: 41)
gcugcugaauuccaugguggc
>GBA ASO 2
(SEQ ID NO: 42)
ggggcauuccucucugcuggg
>GBA ASO 3
(SEQ ID NO: 43)
ugacacccggcucagaggcuu
>GBA ASO 4
(SEQ ID NO: 44)
ugucagagauccggccaugau
>GBA ASO 5
(SEQ ID NO: 45)
ggccugaagcagcagcagucc
>GBA ASO 6
(SEQ ID NO: 46)
agcgccagaagcccaagacac
>GBA ASO 7
(SEQ ID NO: 47)
gcucuuggggaugcaaggucu
>GBA ASO 8
(SEQ ID NO: 48)
cacgacgcugcuguagccgaa
>GBA ASO 9
(SEQ ID NO: 49)
guagguggcauugcacacgca
>GBA ASO 10
(SEQ ID NO: 50)
aggagggucgaagcugucgca
>WPRE nucleic acid sequence
(SEQ ID NO: 51)
Aaucaaccucuggauuacaaaauuugugaaagauugacugguauucuuaa
cuauguugcuccuuuuaccuauguggauacgcugcuuuaaugccuuugua
ucauguauugcuuccguaugguuuuuuuuuuucucuuuaugaggaguugu
ggcccguugucaggcaacguggcguggugugcacuguguuugcugacgca
acccccacugguuggggcauugccaccaccugucagcuccuuuccgggac
uuucgcuuucccccucccuauugccacggggaacucaucgccgccugccu
ugcccgcugcuggacaggggcucggcuguugggcacugacaauuccgugg
uguugucggggaaaucaucguccuuuccuuggcugcucgccuguguugcc
accuggauucugggggacguccuucuguacgucccuucggcccucaaucc
agccuuugggccgccuccccgc
>WPRE ASO 1
(SEQ ID NO: 52)
uuuguaauccagagguugauu
>WPRE ASO 2
(SEQ ID NO: 53)
accagucaaucuuucacaaau
>WPRE ASO 3
(SEQ ID NO: 54)
aggagcaacauaguuaagaau
>WPRE ASO 4
(SEQ ID NO: 55)
agcguauccacauagcguaaa
>WPRE ASO 5
(SEQ ID NO: 56)
augauacaaaggcauuaaagc
>WPRE ASO 6
(SEQ ID NO: 57)
agccauacgggaagcaauagc
>WPRE ASO 7
(SEQ ID NO: 58)
auacaaggaggagaaaaugaa
>WPRE ASO 8
(SEQ ID NO: 59)
aagagacagcaaccaggauuu
>WPRE ASO 9
(SEQ ID NO: 60)
aacgggccacaacuccucaua
>WPRE ASO 10
(SEQ ID NO: 61)
caccacgccacguugccugac
>WPRE ASO 11
(SEQ ID NO: 62)
ugcgucagcaaacacagugca
>WPRE ASO 12
(SEQ ID NO: 63)
aaugccccaaccagugggggu
>WPRE ASO 13
(SEQ ID NO: 64)
aaggagcugacaggugguggc
>WPRE ASO 14
(SEQ ID NO: 65)
ggggaaagcgaaagucccgga
>WPRE ASO 15
(SEQ ID NO: 66)
uuccgccguggcaauagggag
>WPRE ASO 16
(SEQ ID NO: 67)
ggcaaggcaggcggcgaugag
>WPRE ASO 17
(SEQ ID NO: 68)
ccgagccccuguccagcagcg
>WPRE ASO 18
(SEQ ID NO: 69)
ggaauugucagugcccaacag
>WPRE ASO 19
(SEQ ID NO: 70)
ugauuuccccgacaacaccac
>WPRE ASO 20
(SEQ ID NO: 71)
gagcagccaaggaaaggacga
>WPRE ASO 21
(SEQ ID NO: 72)
aauccagguggcaacacaggc
>WPRE ASO 22
(SEQ ID NO: 73)
gcagaaggacgucccgcgcag
>WPRE ASO 23
(SEQ ID NO: 74)
auugagggccgaagggacgua
>WPRE ASO 24
(SEQ ID NO: 75)
gcgggaaggaagguccgcugg
>WPRE ASO 25
(SEQ ID NO: 76)
ccgcagagccggcagcaggcc
>WPRE ASO 26
(SEQ ID NO: 77)
aaggcgaagacgcggaagagg
>WPRE ASO 27
(SEQ ID NO: 78)
gauccgacucgucugagggcg
>WPRE ASO 28
(SEQ ID NO: 79)
cggggaggcggcccaaaggga
> Bovine growth hormone poly-adenylation addition
element
(SEQ ID NO: 80)
cugugccuucuaguugccagccaucuguuuuugcccucccccgugccuuc
cuugacccuggaaggugccacucccacuguccuuuccuaauaaaaugagg
aaauugcaucgcauugucugaguaggugucauucuauucugggggguggg
gggggcaggacagcaagggggaggauugggaagacaauagcaggcaugcu
gggga
>PolyA ASO 1
(SEQ ID NO: 81)
gcuggcaacuagaaggcacag
>PolyA ASO 2
(SEQ ID NO: 82)
gggaggggcaaacaacagaug
>PolyA ASO 3
(SEQ ID NO: 83)
ccagggucaaggaaggcacgg
>PolyA ASO 4
(SEQ ID NO: 84)
ggacagugggaguggcaccuu
>PolyA ASO 5
(SEQ ID NO: 85)
uuuccucauuuuauuaggaaa
>PolyA ASO 6
(SEQ ID NO: 86)
uacucagacaaugcgaugcaa
>PolyA ASO 7
(SEQ ID NO: 87)
cccccagaauagaaugacacc
>PolyA ASO 8
(SEQ ID NO: 88)
ugcuguccugccccaccccac
>PolyA ASO 9
(SEQ ID NO: 89)
ugucuucccaauccucccccu
>PolyA ASO 10
(SEQ ID NO: 90)
ucuccccagcaugccugcuau
>GBA ASO 1 modified
(SEQ ID NO: 91)
mG*mG*mG*mG*mC*A*T*T*C*C*T*C*T*C*T*mG*mC*mU*mG*mG
>GBA ASO 2 modified
(SEQ ID NO: 92)
mU*mG*mC*mA*mG*T*G*T*C*A*G*C*A*G*C*mA*mG*mG*mC*mC
>GBA ASO 3 modified
(SEQ ID NO: 93)
mG*mG*mU*mG*mG*A*G*A*C*A*G*A*G*C*C*mA*mG*mG*mA*mU
>GBA ASO 4 modified
(SEQ ID NO: 94)
mG*mC*mC*mU*mU*C*C*T*C*G*C*T*G*A*A*mG*mU*mA*mG*mC
>GBA ASO 5 modified
(SEQ ID NO: 95)
mC*mG*mC*mA*mG*C*T*G*G*C*C*A*T*G*G*mG*mC*mA*mC*mU
>WPRE ASO 1 modified
(SEQ ID NO: 96)
mC*mU*mU*mU*mC*A*C*A*A*A*T*T*T*T*G*T*mA*mA*mU*
mC*mC
>WPRE ASO 2 modified
(SEQ ID NO: 97)
mG*mG*mC*mA*mU*T*A*A*A*G*C*A*G*C*G*mU*mA*mU*mC*mC
>WPRE ASO 3 modified
(SEQ ID NO: 98)
mC*mC*mC*mC*mG*A*C*A*A*C*A*C*C*A*C*G*G*mA*mA*mU*
mU*mG
>WPRE ASO 4 modified
(SEQ ID NO: 99)
mG*mG*mG*mC*mC*G*A*A*G*G*G*A*C*G*T*mA*mG*mC*mA*mG
>WPRE ASO 5 modified
(SEQ ID NO: 100)
mG*mC*mG*mG*mG*G*A*G*G*C*G*G*C*C*C*A*mA*mA*mG*mG*
mG
>PolyA ASO 1 modified
(SEQ ID NO: 101)
mG*mG*mC*mU*mG*G*C*A*A*C*T*A*G*A*A*mG*mG*mC*mA*mC
>PolyA ASO 2 modified
(SEQ ID NO: 102)
mG*mG*mU*mC*mA*A*G*G*A*A*G*G*C*A*C*mG*mG*mG*mG*mG
>PolyA ASO 3 modified
(SEQ ID NO: 103)
mC*mC*mC*mC*mC*C*A*G*A*A*T*A*G*A*A*T*G*mA*mC*mA*
mC*mC
>PolyA ASO 5 modified
(SEQ ID NO: 104)
mG*mG*mA*mC*mA*mG*mU*mG*mG*mG*mA*mG*mU*mG*mG*mC*
mA*mC*mC
>GFP ASO 1 modified
(SEQ ID NO: 105)
mU*mG*mU*mG*mG*C*C*G*T*T*T*A*C*G*T*mC*mG*mC*mC*mG
>GBA ASO 1 unmodified
(SEQ ID NO: 106)
Ggggcauuccucucugcugg
>GBA ASO 2 unmodified
(SEQ ID NO: 107)
ugcagugucagcagcaggcc
>GBA ASO 3 unmodified
(SEQ ID NO: 108)
gguggagacagagccaggau
>GBA ASO 4 unmodified
(SEQ ID NO: 109)
gccuuccucgcugaaguagc
>GBA ASO 5 unmodified
(SEQ ID NO: 110)
cgcagcuggccaugggcacu
>WPRE ASO 1 unmodified
(SEQ ID NO: 111)
Cuuucacaaauuuuguaaucc
>WPRE ASO 2 unmodified
(SEQ ID NO: 112)
Ggcauuaaagcagcguaucc
>WPRE ASO 3 unmodified
(SEQ ID NO: 113)
Ccccgacaacaccacggaauug
>WPRE ASO 4 unmodified
(SEQ ID NO: 114)
Gggccgaagggacguagcag
>WPRE ASO 5 unmodified
(SEQ ID NO: 115)
Gcggggaggcggcccaaaggg
>PolyA ASO 1 unmodified
(SEQ ID NO: 116)
ggcuggcaacuagaaggcac
>PolyA ASO 2 unmodified
(SEQ ID NO: 117)
ggucaaggaaggcacggggg
>PolyA ASO 3 unmodified
(SEQ ID NO: 118)
ccccccagaauagaaugacacc
>PolyA ASO 4 unmodified
(SEQ ID NO: 119)
ccauagagcccaccgcaucccc
>PolyA ASO 5 unmodified
(SEQ ID NO: 120)
ggacagugggaguggcacc

What is claimed is:

1. A method for modulating expression of a transgene in a cell, the method comprising contacting a cell containing an rAAV vector comprising a transgene flanked by AAV inverted terminal repeats (ITRs) with one or more antisense oligonucleotides (ASOs) that specifically bind to at least one of the AAV ITRs, wherein binding of the one or more ASOs to the AAV ITR results in altered expression of the transgene relative to a cell that does not contain the one or more ASOs.

2. The method of claim 1, wherein each of the one or more ASOs ranges from about nucleotides to about 30 nucleotides in length.

3. The method of claim 1 or claim 2, wherein each of the ASOs comprises one or more chemical modification.

4. The method of claim 3, wherein each of the one or more chemical modifications is selected from a nucleobase modification or a backbone modification.

5. The method of claim 4, wherein all the nucleobases and/or the entire backbone of each of the ASOs are modified.

6. The method of claim 4 or 5, wherein the nucleobase modification comprises a 2′-O-methyl (2′OMe) modification.

7. The method of claim 4 or 5, wherein the backbone modification comprises a phosphorothioate linkage.

8. The method of claim 4 or 5, wherein an ASO comprises one or more locked nucleic acids (LNAs).

9. The method of any one of claims 1 to 8, wherein the AAV ITR is an AAV2 ITR.

10. The method of claim 9, wherein the AAV2 ITR comprises a nucleic acid sequence that is at least 90% identical to the nucleic acid sequence set forth in SEQ ID NO: 1 or the complement thereof.

11. The method of claim 10, wherein the AAV2 ITR consists of the nucleic acid sequence set forth in SEQ ID NO: 1 or the complement thereof.

12. The method of any one of claims 1 to 11, wherein the ASO binds to at least three contiguous nucleotides of the AAV ITR.

13. The method of any one of claims 1 to 12, wherein the at least one ASO comprises a nucleic acid sequence that is at least 90% identical to the sequence set forth in any one of SEQ ID NOs: 2-8, or a complement thereof.

14. The method of any one of claims 1 to 13, wherein the altered expression is increased expression of the transgene.

15. The method of any one of claims 1 to 13, wherein the altered expression is decreased expression of the transgene.

16. The method of any one of claims 1 to 15, wherein the cell is a mammalian cell, optionally wherein the mammalian cell is a human cell.

17. The method of any one of claims 1 to 16, wherein the cell is in a subject.

18. The method of any one of claims 1 to 17, wherein the transgene is a therapeutic protein.

19. The method of claim 18, wherein the therapeutic protein is β-glucocerebrosidase (GBA).

20. The method of claim 19, wherein the GBA is encoded by a codon-optimized nucleic acid sequence.

21. The method of claim 19 or 20, wherein the transgene encoding GBA comprises the nucleic acid sequence set forth in SEQ ID NO: 40 or the complement thereof.

22. The method of any one of claims 1 to 21, wherein the rAAV vector comprises the nucleic acid sequences set forth in SEQ ID NOs: 1, 9, 25, 40, 51, and 80.

23. A method for modulating expression of a transgene in a cell, the method comprising contacting a cell containing an rAAV vector comprising a transgene with one or more antisense oligonucleotides (ASOs) that specifically bind to a transcriptional control region sequence of the transgene, wherein binding of the one or more ASOs to the transcriptional control region sequence results in altered expression of the transgene relative to a cell that does not contain the one or more ASOs.

24. The method of claim 23, wherein each of the one or more ASOs ranges from about 10 nucleotides to about 30 nucleotides in length.

25. The method of claim 23 or claim 24, wherein each of the ASOs comprises one or more chemical modification.

26. The method of claim 25, wherein each of the one or more chemical modifications is selected from a nucleobase modification or a backbone modification.

27. The method of claim 26, wherein all the nucleobases and/or the entire backbone of each of the ASOs are modified.

28. The method of claim 26 or 27, wherein the nucleobase modification comprises a 2′-O-methyl (2′OMe) modification.

29. The method of claim 26 or 27, wherein the backbone modification comprises a phosphorothioate linkage.

30. The method of claim 26 or 27, wherein an ASO comprises one or more locked nucleic acids (LNAs).

31. The method of any one of claims 23 to 30, wherein the transcriptional control region sequence comprises an enhancer sequence and/or a promoter sequence.

32. The method of claim 31, wherein the enhancer sequence is a cytomegalovirus (CMV) enhancer sequence and/or the promoter sequence is a chicken beta-actin (CBA) promoter sequence.

33. The method of claim 32, wherein the (CMV) enhancer sequence comprises a nucleic acid sequence that is at least 90% identical to the nucleic acid sequence set forth in SEQ ID NO: 9 or the complement thereof, and/or the chicken beta-actin (CBA) promoter sequence comprises a nucleic acid sequence that is at least 90% identical to the nucleic acid sequence set forth in SEQ ID NO: 25 or the complement thereof.

34. The method of any one of claims 23 to 33, wherein the ASO binds to at least three contiguous nucleotides of the transcriptional control region sequence.

35. The method of any one of claims 23 to 34, wherein the at least one ASO comprises a nucleic acid sequence that is at least 90% identical to the sequence set forth in any one of SEQ ID NOs: 10-24 and 26-39, or a complement thereof.

36. The method of claim 35, wherein at the at least one ASO comprises one or more chemical modification selected from a nucleobase modification or a backbone modification.

37. The method of claim 36, wherein each of the one or more chemical modifications is selected from a nucleobase modification or a backbone modification.

38. The method of claim 37, wherein all the nucleobases and/or the entire backbone of each of the ASOs are modified.

39. The method of claim 37 or 38, wherein the nucleobase modification comprises a 2′-O-methyl (2′OMe) modification.

40. The method of claim 37 or 38, wherein the backbone modification comprises a phosphorothioate linkage.

41. The method of claim 37 or 38, wherein an ASO comprises one or more locked nucleic acids (LNAs).

42. The method of any one of claims 23 to 41, wherein the altered expression is increased expression of the transgene.

43. The method of any one of claims 23 to 41, wherein the altered expression is decreased expression of the transgene.

44. The method of any one of claims 23 to 43, wherein the cell is a mammalian cell, optionally wherein the mammalian cell is a human cell.

45. The method of any one of claims 23 to 44, wherein the cell is in a subject.

46. The method of any one of claims 23 to 45, wherein the transgene is a therapeutic protein.

47. The method of claim 46, wherein the therapeutic protein is β-glucocerebrosidase (GBA).

48. The method of claim 47, wherein the GBA is encoded by a codon-optimized nucleic acid sequence.

49. The method of claim 47 or 48, wherein the transgene encoding GBA comprises the nucleic acid sequence set forth in SEQ ID NO: 40 or the complement thereof.

50. The method of any one of claims 35 to 49, wherein the rAAV vector comprises the nucleic acid sequences set forth in SEQ ID NOs: 1, 9, 25, 40, 51, and 80.

51. A method for modulating expression of a transgene in a cell, the method comprising contacting a cell containing an rAAV vector comprising a transgene with one or more antisense oligonucleotides (ASOs) that specifically bind to a protein coding region of an mRNA transcribed from the transgene, wherein binding of the one or more ASOs to the protein coding region results in altered expression of the transgene relative to a cell that does not contain the one or more ASOs.

52. The method of claim 51, wherein each of the one or more ASOs ranges from about 10 nucleotides to about 30 nucleotides in length.

53. The method of claim 51 or claim 52, wherein each of the ASOs comprises one or more chemical modification.

54. The method of claim 53, wherein each of the one or more chemical modifications is selected from a nucleobase modification or a backbone modification.

55. The method of claim 54, wherein all the nucleobases and/or the entire backbone of each of the ASOs are modified.

56. The method of claim 54 or 55, wherein the nucleobase modification comprises a 2′-O-methyl (2′OMe) modification.

57. The method of claim 54 or 55, wherein the backbone modification comprises a phosphorothioate linkage.

58. The method of claim 54 or 55, wherein an ASO comprises one or more locked nucleic acids (LNAs).

59. The method of any one of claims 51 to 58, wherein the ASO comprises a gapmer structure.

60. The method of any one of claims 51 to 59, wherein the ASO binds to at least three contiguous nucleotides of the protein coding region.

61. The method of any one of claims 51 to 60, wherein the at least one ASO comprises a nucleic acid sequence that is at least 90% identical to the sequence set forth in any one of SEQ ID NOs: 41-50, any one of SEQ ID NOs: 91-95, or any one of SEQ ID NOs: 106-110, or a complement thereof.

62. The method of any one of claims 51 to 61, wherein the protein coding region encodes a β-glucocerebrosidase (GBA) protein.

63. The method of claim 62, wherein the protein coding region comprises a nucleic acid sequence that is at least 90% identical to the nucleic acid sequence set forth in SEQ ID NO: 40 or the complement thereof.

64. The method of any one of claims 51 to 63, wherein the altered expression is increased expression of the transgene.

65. The method of any one of claims 51 to 63, wherein the altered expression is decreased expression of the transgene.

66. The method of any one of claims 51 to 65, wherein the expression modulation occurs, irrespective of the nature of the expressed transgene.

67. The method of any one of claims 51 to 66, the one or more ASOs are delivered to the cell at the same time of transgene transfection.

68. The method of any one of claims 51 to 66, the one or more ASOs are delivered to the cell several hours, for example 3 hours, after the cell is transfected with the plasmid comprising the rAAV vector encoding the transgene.

69. The method of any one of claims 51 to 66, the one or more ASOs is delivered to the cell several weeks after the cell is transfected with the plasmid comprising the rAAV vector encoding the transgene.

70. The method of claim 65, wherein the decreased expression of the transgene results from RNaseH-mediated degradation of mRNA transcripts bound by the one or more ASOs.

71. The method of any one of claims 51 to 70, wherein the cell is a mammalian cell, optionally wherein the mammalian cell is a human cell.

72. The method of any one of claims 51 to 71, wherein the cell is in a subject.

73. The method of any one of claims 51 to 72, wherein the rAAV vector comprises the nucleic acid sequences set forth in SEQ ID NOs: 1, 9, 25, 40, 51, and 80.

74. A method for modulating expression of a transgene in a cell, the method comprising contacting a cell containing an rAAV vector comprising a transgene with one or more antisense oligonucleotides (ASOs) that specifically bind to a woodchuck post-translational regulatory element (WPRE) of an mRNA transcribed from the transgene, wherein binding of the one or more ASOs to the WPRE results in altered expression of the transgene relative to a cell that does not contain the one or more ASOs.

75. The method of claim 74, wherein each of the one or more ASOs ranges from about 10 nucleotides to about 30 nucleotides in length.

76. The method of claim 74 or claim 75, wherein each of the ASOs comprises one or more chemical modification.

77. The method of claim 76, wherein each of the one or more chemical modifications is selected from a nucleobase modification or a backbone modification.

78. The method of claim 77, wherein all the nucleobases and/or the entire backbone of each of the ASOs are modified.

79. The method of claim 77 or 78, wherein the nucleobase modification comprises a 2′-O-methyl (2′OMe) modification.

80. The method of claim 77 or 78, wherein the backbone modification comprises a phosphorothioate linkage.

81. The method of claim 77 or 78, wherein an ASO comprises one or more locked nucleic acids (LNAs).

82. The method of claim 80 or 81, wherein the WPRE comprises a nucleic acid sequence that is at least 90% identical to the nucleic acid sequence set forth in SEQ ID NO: 51 or the complement thereof.

83. The method of any one of claims 74 to 82, wherein the ASO binds to at least three contiguous nucleotides of the WPRE sequence.

84. The method of any one of claims 74 to 83, wherein the at least one ASO comprises a nucleic acid sequence that is at least 90% identical to the sequence set forth in any one of SEQ ID NOs: 52-79, any one of SEQ ID NOs: 96-100, or any one of SEQ ID NOs: 111-115, or a complement thereof.

85. The method of any one of claims 74 to 84, wherein the altered expression is increased expression of the transgene.

86. The method of any one of claims 74 to 85, wherein the altered expression is decreased expression of the transgene.

87. The method of any one of claims 74 to 86, wherein the cell is a mammalian cell, optionally wherein the mammalian cell is a human cell.

88. The method of any one of claims 74 to 87, wherein the cell is in a subject.

89. The method of any one of claims 74 to 88, wherein the transgene is a therapeutic protein.

90. The method of claim 89, wherein the therapeutic protein is β-glucocerebrosidase (GBA).

91. The method of claim 90, wherein the GBA is encoded by a codon-optimized nucleic acid sequence.

92. The method of claim 90 or 91, wherein the transgene encoding GBA comprises the nucleic acid sequence set forth in SEQ ID NO: 40 or the complement thereof.

93. The method of any one of claims 74 to 92, wherein the rAAV vector comprises the nucleic acid sequences set forth in SEQ ID NOs: 1, 9, 25, 40, 51, and 80.

94. A method for modulating expression of a transgene in a cell, the method comprising contacting a cell containing an rAAV vector comprising a transgene with one or more antisense oligonucleotides (ASOs) that specifically bind to a polyadenylation element of an mRNA transcribed from the transgene, wherein binding of the one or more ASOs to the polyadenylation element results in altered expression of the transgene relative to a cell that does not contain the one or more ASOs.

95. The method of claim 94, wherein each of the one or more ASOs ranges from about 10 nucleotides to about 30 nucleotides in length.

96. The method of claim 94 or claim 95, wherein each of the ASOs comprises one or more chemical modification.

97. The method of claim 96, wherein each of the one or more chemical modifications is selected from a nucleobase modification or a backbone modification.

98. The method of claim 97, wherein all the nucleobases and/or the entire backbone of each of the ASOs are modified.

99. The method of claim 96 or 97, wherein the nucleobase modification comprises a 2′-O-methyl (2′OMe) modification.

100. The method of claim 96 or 97, wherein the backbone modification comprises a phosphorothioate linkage.

101. The method of claim 96 or 97, wherein an ASO comprises one or more locked nucleic acids (LNAs).

102. The method of any one of claims 94 to 101, wherein the ASO comprises a gapmer structure.

103. The method of any one of claims 94 to 102, wherein the ASO binds to at least three contiguous nucleotides of the polyadenylation element.

104. The method of any one of claims 94 to 103, wherein the polyadenylation element comprises the nucleic acid sequence set forth in SEQ ID NO: 80 or the complement thereof.

105. The method of any one of claims 94 to 104, wherein the at least one ASO comprises a nucleic acid sequence that is at least 90% identical to the sequence set forth in any one of SEQ ID NOs: 81-90, any one of SEQ ID NOs: 101-104, or any one of SEQ ID NOs: 116-120, or a complement thereof.

106. The method of any one of claims 94 to 105, wherein the altered expression is increased expression of the transgene.

107. The method of any one of claims 94 to 106, wherein the altered expression is decreased expression of the transgene.

108. The method of claim 107, wherein the decreased expression of the transgene results from RNaseH-mediated degradation of mRNA transcripts bound by the one or more ASOs.

109. The method of any one of claims 94 to 108, wherein the cell is a mammalian cell, optionally wherein the mammalian cell is a human cell.

110. The method of any one of claims 94 to 109, wherein the cell is in a subject.

111. The method of any one of claims 94 to 110, wherein the transgene is a therapeutic protein.

112. The method of claim 111, wherein the therapeutic protein is β-glucocerebrosidase (GBA).

113. The method of claim 112, wherein the GBA is encoded by a codon-optimized nucleic acid sequence.

114. The method of claim 112-113, wherein the transgene encoding GBA comprises the nucleic acid sequence set forth in SEQ ID NO: 40 or the complement thereof.

115. The method of any one of claims 94 to 114, wherein the rAAV vector comprises the nucleic acid sequences set forth in SEQ ID NOs: 1, 9, 25, 40, 51, and 80.

116. An isolated nucleic acid comprising the sequence set forth in any one of SEQ ID NOs: 2-8, 10-24, 26-39, 41-50, 52-79, 81-120, or a complement thereof.

117. The isolated nucleic acid of claim 116, wherein the isolated nucleic acid comprises one or more chemical modifications.

118. The isolated nucleic acid of claim 116 or 117, wherein the one or more chemical modifications comprises a 2′-O-methyl (2′OMe) modification, a phosphorothioate linkage, a locked nucleic acid (LNA), or any combination of the foregoing.

119. The isolated nucleic acid of any one of claims 116 to 118, wherein the isolated nucleic acid is an antisense oligonucleotide (ASO).

120. The isolated nucleic acid of claim 119, wherein all the nucleobases and/or the entire backbone of the ASO are modified.

121. The isolated nucleic acid of any one of claims 116 to 120, wherein the isolated nucleic acid has a gapmer structure.


SEQUENCE LISTING (120 sequences)

SEQ ID NO: 1 - DNA, 141 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 19 bp, Artificial sequence
  Synthetic
gcgcgcagctgcctgcagg

SEQ ID NO: 3 - DNA, 20 bp, Artificial sequence
  Synthetic
cggcctcagtgagcgagcga

SEQ ID NO: 4 - DNA, 20 bp, Artificial sequence
  Synthetic
acgcccgggctttgcccggg

SEQ ID NO: 5 - DNA, 21 bp, Artificial sequence
  Synthetic
cgggcgaccaaaggtcgcccg

SEQ ID NO: 6 - DNA, 20 bp, Artificial sequence
  Synthetic
gctcgctcgctcactgaggc

SEQ ID NO: 7 - DNA, 20 bp, Artificial sequence
  Synthetic
tggccactccctctctgcgc

SEQ ID NO: 8 - DNA, 21 bp, Artificial sequence
  Synthetic
aggaacccctagtgatggagt

SEQ ID NO: 9 - DNA, 304 bp, Artificial sequence
  Synthetic
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 10 - DNA, 21 bp, Artificial sequence
  Synthetic
tttaccgtaagttatgtaacg

SEQ ID NO: 11 - DNA, 21 bp, Artificial sequence
  Synthetic
ggcggtcagccaggcgggcca

SEQ ID NO: 12 - DNA, 21 bp, Artificial sequence
  Synthetic
gtcaatgggcgggggtcgttg

SEQ ID NO: 13 - DNA, 21 bp, Artificial sequence
  Synthetic
ggaacatacgtcattattgac

SEQ ID NO: 14 - DNA, 21 bp, Artificial sequence
  Synthetic
gtccctattggcgttactatg

SEQ ID NO: 15 - DNA, 21 bp, Artificial sequence
  Synthetic
acccattgacgtcaatggaaa

SEQ ID NO: 16 - DNA, 21 bp, Artificial sequence
  Synthetic
gcagtttaccgtaaatactcc

SEQ ID NO: 17 - DNA, 21 bp, Artificial sequence
  Synthetic
acttgatgtactgccaagtgg

SEQ ID NO: 18 - DNA, 21 bp, Artificial sequence
  Synthetic
ggcgtacttggcatatgatac

SEQ ID NO: 19 - DNA, 21 bp, Artificial sequence
  Synthetic
ccgtcattgacgtcaataggg

SEQ ID NO: 20 - DNA, 21 bp, Artificial sequence
  Synthetic
taatgccaggcgggccattta

SEQ ID NO: 21 - DNA, 21 bp, Artificial sequence
  Synthetic
cataaggtcatgtactgggca

SEQ ID NO: 22 - DNA, 21 bp, Artificial sequence
  Synthetic
tactgccaagtaggaaagtcc

SEQ ID NO: 23 - DNA, 21 bp, Artificial sequence
  Synthetic
gcgatgactaatacgtagatg

SEQ ID NO: 24 - DNA, 19 bp, Artificial sequence
  Synthetic
ccatggtaatagcgatgac

SEQ ID NO: 25 - DNA, 278 bp, Artificial sequence
  Synthetic
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 26 - DNA, 21 bp, Artificial sequence
  Synthetic
agaacgtggggctcacctcga

SEQ ID NO: 27 - DNA, 21 bp, Artificial sequence
  Synthetic
gggagatggggagagtgaagc

SEQ ID NO: 28 - DNA, 21 bp, Artificial sequence
  Synthetic
aaattgggggtggggaggggg

SEQ ID NO: 29 - DNA, 21 bp, Artificial sequence
  Synthetic
attaaaaaataaataaataca

SEQ ID NO: 30 - DNA, 21 bp, Artificial sequence
  Synthetic
cccccatcgctgcacaaaata

SEQ ID NO: 31 - DNA, 21 bp, Artificial sequence
  Synthetic
cgcgccccccccccccccccg

SEQ ID NO: 32 - DNA, 21 bp, Artificial sequence
  Synthetic
cccgccccgccccgcctggcg

SEQ ID NO: 33 - DNA, 21 bp, Artificial sequence
  Synthetic
tcgccccgccccgcccctcgc

SEQ ID NO: 34 - DNA, 21 bp, Artificial sequence
  Synthetic
ctgccgccgcacctctccgcc

SEQ ID NO: 35 - DNA, 21 bp, Artificial sequence
  Synthetic
ggagcgcgccgctctgattgg

SEQ ID NO: 36 - DNA, 21 bp, Artificial sequence
  Synthetic
tcgccataaaaggaaactttc

SEQ ID NO: 37 - DNA, 21 bp, Artificial sequence
  Synthetic
agggccgccgccgccgccgcc

SEQ ID NO: 38 - DNA, 21 bp, Artificial sequence
  Synthetic
gccgcgcgcttcgctttttat

SEQ ID NO: 39 - DNA, 21 bp, Artificial sequence
  Synthetic
agcgcgcagcgactcccgccc

SEQ ID NO: 40 - RNA, 1611 nt, Artificial sequence
  Synthetic
auggaauucagcagccccagcagagaggaaugccccaagccucugagccgggugucaaucauggccggaucucugacaggacugcugcugcuucaggccgugucuugggcuucuggcgcuagaccuugcauccccaagagcuucggcuacagcagcgucgugugcgugugcaaugccaccuacugcgacagcuucgacccuccuaccuuuccugcucugggcaccuucagcagauacgagagcaccagauccggcagacggauggaacugagcaugggacccauccaggccaaucacacaggcacuggccugcugcugacacugcagccugagcagaaauuccagaaagugaaaggcuucggcggagccaugacagaugccgccgcucugaauauccuggcucugucuccaccagcucagaaccugcugcucaagagcuacuucagcgaggaaggcaucggcuacaacaucaucagagugcccauggccagcugcgacuucagcaucaggaccuacaccuacgccgacacacccgacgauuuccagcugcacaacuucagccugccugaagaggacaccaagcugaagaucccucugauccacagagcccugcagcuggcacaaagacccgugucacugcuggccucuccauggacaucucccaccuggcugaaaacaaauggcgccgugaauggcaagggcagccugaaaggccaaccuggcgacaucuaccaccagaccugggccagauacuucgugaaguuccuggacgccuaugccgagcacaagcugcaguuuugggccgugacagccgagaacgaaccuucugcuggacugcugagcggcuaccccuuucagugccugggcuuuacacccgagcaccagcgggacuuuaucgcccgugaucugggacccacacuggccaauagcacccaccauaaugugcggcugcugaugcuggacgaccagagacugcuucugccccacugggcuaaaguggugcugacagauccugaggccgccaaauacgugcacggaaucgccgugcacugguaucuggacuuucuggccccugccaaggccacacugggagagacacacagacuguuccccaacaccaugcuguucgccagcgaagccugugugggcagcaaguuuugggaacagagcgugcggcucggcagcugggauagaggcaugcaguacagccacagcaucaucaccaaccugcuguaccacgucgucggcuggaccgacuggaaucuggcccugaauccugaaggcggcccuaacuggguccgaaacuucguggacagccccaucaucguggacaucaccaaggacaccuucuacaagcagcccauguucuaccaccugggacacuucagcaaguucauccccgagggcucucagcgcguuggacugguggcuucccagaagaacgaucuggacgccguggcucugaugcacccugauggaucugcugugguggugguccugaaccgcagcagcaaagaugugccccugaccaucaaggaucccgccgugggauuccuggaaacaaucagcccuggcuacuccauccacaccuaccuguggcguagacaguga

SEQ ID NO: 41 - RNA, 21 nt, Artificial sequence
  Synthetic
gcugcugaauuccaugguggc

SEQ ID NO: 42 - RNA, 21 nt, Artificial sequence
  Synthetic
ggggcauuccucucugcuggg

SEQ ID NO: 43 - RNA, 21 nt, Artificial sequence
  Synthetic
ugacacccggcucagaggcuu

SEQ ID NO: 44 - RNA, 21 nt, Artificial sequence
  Synthetic
ugucagagauccggccaugau

SEQ ID NO: 45 - RNA, 21 nt, Artificial sequence
  Synthetic
ggccugaagcagcagcagucc

SEQ ID NO: 46 - RNA, 21 nt, Artificial sequence
  Synthetic
agcgccagaagcccaagacac

SEQ ID NO: 47 - RNA, 21 nt, Artificial sequence
  Synthetic
gcucuuggggaugcaaggucu

SEQ ID NO: 48 - RNA, 21 nt, Artificial sequence
  Synthetic
cacgacgcugcuguagccgaa

SEQ ID NO: 49 - RNA, 21 nt, Artificial sequence
  Synthetic
guagguggcauugcacacgca

SEQ ID NO: 50 - RNA, 21 nt, Artificial sequence
  Synthetic
aggagggucgaagcugucgca

SEQ ID NO: 51 - RNA, 589 nt, Artificial sequence
  Synthetic
aaucaaccucuggauuacaaaauuugugaaagauugacugguauucuuaacuauguugcuccuuuuacgcuauguggauacgcugcuuuaaugccuuuguaucaugcuauugcuucccguauggcuuucauuuucuccuccuuguauaaauccugguugcugucucuuuaugaggaguuguggcccguugucaggcaacguggcguggugugcacuguguuugcugacgcaacccccacugguuggggcauugccaccaccugucagcuccuuuccgggacuuucgcuuucccccucccuauugccacggcggaacucaucgccgccugccuugcccgcugcuggacaggggcucggcuguugggcacugacaauuccgugguguugucggggaaaucaucguccuuuccuuggcugcucgccuguguugccaccuggauucugcgcgggacguccuucugcuacgucccuucggcccucaauccagcggaccuuccuucccgcggccugcugccggcucugcggccucuuccgcgucuucgccuucgcccucagacgagucggaucucccuuugggccgccuccccgc

SEQ ID NO: 52 - RNA, 21 nt, Artificial sequence
  Synthetic
uuuguaauccagagguugauu

SEQ ID NO: 53 - RNA, 21 nt, Artificial sequence
  Synthetic
accagucaaucuuucacaaau

SEQ ID NO: 54 - RNA, 21 nt, Artificial sequence
  Synthetic
aggagcaacauaguuaagaau

SEQ ID NO: 55 - RNA, 21 nt, Artificial sequence
  Synthetic
agcguauccacauagcguaaa

SEQ ID NO: 56 - RNA, 21 nt, Artificial sequence
  Synthetic
augauacaaaggcauuaaagc

SEQ ID NO: 57 - RNA, 21 nt, Artificial sequence
  Synthetic
agccauacgggaagcaauagc

SEQ ID NO: 58 - RNA, 21 nt, Artificial sequence
  Synthetic
auacaaggaggagaaaaugaa

SEQ ID NO: 59 - RNA, 21 nt, Artificial sequence
  Synthetic
aagagacagcaaccaggauuu

SEQ ID NO: 60 - RNA, 21 nt, Artificial sequence
  Synthetic
aacgggccacaacuccucaua

SEQ ID NO: 61 - RNA, 21 nt, Artificial sequence
  Synthetic
caccacgccacguugccugac

SEQ ID NO: 62 - RNA, 21 nt, Artificial sequence
  Synthetic
ugcgucagcaaacacagugca

SEQ ID NO: 63 - RNA, 21 nt, Artificial sequence
  Synthetic
aaugccccaaccagugggggu

SEQ ID NO: 64 - RNA, 21 nt, Artificial sequence
  Synthetic
aaggagcugacaggugguggc

SEQ ID NO: 65 - RNA, 21 nt, Artificial sequence
  Synthetic
ggggaaagcgaaagucccgga

SEQ ID NO: 66 - RNA, 21 nt, Artificial sequence
  Synthetic
uuccgccguggcaauagggag

SEQ ID NO: 67 - RNA, 21 nt, Artificial sequence
  Synthetic
ggcaaggcaggcggcgaugag

SEQ ID NO: 68 - RNA, 21 nt, Artificial sequence
  Synthetic
ccgagccccuguccagcagcg

SEQ ID NO: 69 - RNA, 21 nt, Artificial sequence
  Synthetic
ggaauugucagugcccaacag

SEQ ID NO: 70 - RNA, 21 nt, Artificial sequence
  Synthetic
ugauuuccccgacaacaccac

SEQ ID NO: 71 - RNA, 21 nt, Artificial sequence
  Synthetic
gagcagccaaggaaaggacga

SEQ ID NO: 72 - RNA, 21 nt, Artificial sequence
  Synthetic
aauccagguggcaacacaggc

SEQ ID NO: 73 - RNA, 21 nt, Artificial sequence
  Synthetic
gcagaaggacgucccgcgcag

SEQ ID NO: 74 - RNA, 21 nt, Artificial sequence
  Synthetic
auugagggccgaagggacgua

SEQ ID NO: 75 - RNA, 21 nt, Artificial sequence
  Synthetic
gcgggaaggaagguccgcugg

SEQ ID NO: 76 - RNA, 21 nt, Artificial sequence
  Synthetic
ccgcagagccggcagcaggcc

SEQ ID NO: 77 - RNA, 21 nt, Artificial sequence
  Synthetic
aaggcgaagacgcggaagagg

SEQ ID NO: 78 - RNA, 21 nt, Artificial sequence
  Synthetic
gauccgacucgucugagggcg

SEQ ID NO: 79 - RNA, 21 nt, Artificial sequence
  Synthetic
cggggaggcggcccaaaggga

SEQ ID NO: 80 - RNA, 208 nt, Artificial sequence
  Synthetic
cugugccuucuaguugccagccaucuguuguuugccccucccccgugccuuccuugacccuggaaggugccacucccacuguccuuuccuaauaaaaugaggaaauugcaucgcauugucugaguaggugucauucuauucuggggggugggguggggcaggacagcaagggggaggauugggaagacaauagcaggcaugcugggga

SEQ ID NO: 81 - RNA, 21 nt, Artificial sequence
  Synthetic
gcuggcaacuagaaggcacag

SEQ ID NO: 82 - RNA, 21 nt, Artificial sequence
  Synthetic
gggaggggcaaacaacagaug

SEQ ID NO: 83 - RNA, 21 nt, Artificial sequence
  Synthetic
ccagggucaaggaaggcacgg

SEQ ID NO: 84 - RNA, 21 nt, Artificial sequence
  Synthetic
ggacagugggaguggcaccuu

SEQ ID NO: 85 - RNA, 21 nt, Artificial sequence
  Synthetic
uuuccucauuuuauuaggaaa

SEQ ID NO: 86 - RNA, 21 nt, Artificial sequence
  Synthetic
uacucagacaaugcgaugcaa

SEQ ID NO: 87 - RNA, 21 nt, Artificial sequence
  Synthetic
cccccagaauagaaugacacc

SEQ ID NO: 88 - RNA, 21 nt, Artificial sequence
  Synthetic
ugcuguccugccccaccccac

SEQ ID NO: 89 - RNA, 21 nt, Artificial sequence
  Synthetic
ugucuucccaauccucccccu

SEQ ID NO: 90 - RNA, 21 nt, Artificial sequence
  Synthetic
ucuccccagcaugccugcuau

SEQ ID NO: 91 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
ggggcattcctctctgcugg

SEQ ID NO: 92 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
ugcagtgtcagcagcaggcc

SEQ ID NO: 93 - RNA, 20 nt, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
gguggagacagagccaggau

SEQ ID NO: 94 - DNA, 20 bp, Artificial sequence
  Synthetic
  modified_base (1)..(5): may be modified by 2′-O-methyl
  modified_base (16)..(20): may be modified by 2′-O-methyl
  misc_feature (16)..(20): phosphorothioate linkage
gccuucctcgctgaaguagc

SEQ ID NO: 95 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
cgcagctggccatgggcacu

SEQ ID NO: 96 - DNA, 21 bp, Artificial sequence
  Synthetic
  modified_base (1)..(5): may be modified by 2′-O-methyl
  misc_feature (1)..(21): phosphorothioate linkage
  modified_base (17)..(21): may be modified by 2′-O-methyl
cuuucacaaattttgtaaucc

SEQ ID NO: 97 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
ggcautaaagcagcguaucc

SEQ ID NO: 98 - RNA, 22 nt, Artificial sequence
  Synthetic
  modified_base (1)..(5): may be modified by 2′-O-methyl
  misc_feature (1)..(22): phosphorothioate linkage
  modified_base (18)..(22): may be modified by 2′-O-methyl
ccccgacaacaccacggaauug

SEQ ID NO: 99 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
gggccgaagggacgtagcag

SEQ ID NO: 100 - DNA, 21 bp, Artificial sequence
  Synthetic
  modified_base (1)..(5): may be modified by 2′-O-methyl
  misc_feature (1)..(21): phosphorothioate linkage
  modified_base (17)..(21): may be modified by 2′-O-methyl
gcggggaggcggcccaaaggg

SEQ ID NO: 101 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
ggcuggcaactagaaggcac

SEQ ID NO: 102 - RNA, 20 nt, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
ggucaaggaaggcacggggg

SEQ ID NO: 103 - DNA, 22 bp, Artificial sequence
  Synthetic
  modified_base (1)..(5): may be modified by 2′-O-methyl
  misc_feature (1)..(22): phosphorothioate linkage
  modified_base (18)..(22): may be modified by 2′-O-methyl
ccccccagaatagaatgacacc

SEQ ID NO: 104 - RNA, 19 nt, Artificial sequence
  Synthetic
  modified_base (1)..(19): may be modified by 2′-O-methyl
  misc_feature (1)..(19): phosphorothioate linkage
ggacagugggaguggcacc

SEQ ID NO: 105 - DNA, 20 bp, Artificial sequence
  Synthetic
  modified_base (1)..(5): may be modified by 2′-O-methyl
  misc_feature (1)..(20): phosphorothioate linkage
  modified_base (16)..(20): may be modified by 2′-O-methyl
uguggccgtttacgtcgccg

SEQ ID NO: 106 - RNA, 20 nt, Artificial sequence
  Synthetic
ggggcauuccucucugcugg

SEQ ID NO: 107 - RNA, 20 nt, Artificial sequence
  Synthetic
ugcagugucagcagcaggcc

SEQ ID NO: 108 - RNA, 20 nt, Artificial sequence
  Synthetic
gguggagacagagccaggau

SEQ ID NO: 109 - RNA, 20 nt, Artificial sequence
  Synthetic
gccuuccucgcugaaguagc

SEQ ID NO: 110 - RNA, 20 nt, Artificial sequence
  Synthetic
cgcagcuggccaugggcacu

SEQ ID NO: 111 - RNA, 21 nt, Artificial sequence
  Synthetic
cuuucacaaauuuuguaaucc

SEQ ID NO: 112 - RNA, 20 nt, Artificial sequence
  Synthetic
ggcauuaaagcagcguaucc

SEQ ID NO: 113 - RNA, 22 nt, Artificial sequence
  Synthetic
ccccgacaacaccacggaauug

SEQ ID NO: 114 - RNA, 20 nt, Artificial sequence
  Synthetic
gggccgaagggacguagcag

SEQ ID NO: 115 - RNA, 21 nt, Artificial sequence
  Synthetic
gcggggaggcggcccaaaggg

SEQ ID NO: 116 - RNA, 20 nt, Artificial sequence
  Synthetic
ggcuggcaacuagaaggcac

SEQ ID NO: 117 - RNA, 20 nt, Artificial sequence
  Synthetic
ggucaaggaaggcacggggg

SEQ ID NO: 118 - RNA, 22 nt, Artificial sequence
  Synthetic
ccccccagaauagaaugacacc

SEQ ID NO: 119 - RNA, 22 nt, Artificial sequence
  Synthetic
ccauagagcccaccgcaucccc

SEQ ID NO: 120 - RNA, 19 nt, Artificial sequence
  Synthetic
ggacagugggaguggcacc